(19) United States
(12) Patent Application Publication
(10) Pub. No.: US 2025/0275951 A1
(43) Pub. Date: Sep. 4, 2025

(54) DRUGS TARGETING INFLAMMATION FOR THE TREATMENT OF OSTEOARTHRITIS AND OTHER INFLAMMATORY DISEASES

(71) Applicant: The Board of Trustees of the Leland Stanford Junior University, Stanford, CA (US)

(72) Inventors: William H. Robinson, Palo Alto, CA (US); Qian Wang, Palo Alto, CA (US); Matthew C. Baker, Menlo Park, CA (US)

(21) Appl. No.: 18/034,649

(22) PCT Filed: Nov. 1, 2021
(86) PCT No.: PCT/US2021/057612

§ 371 Date: Apr. 28, 2023

Related U.S. Application Data
(60) Provisional application No. 63/108,072, filed on Oct. 30, 2020.

(51) Int. Cl. A61K 31/4545; A61K 31/135; A61K 31/155; A61K 31/216; A61K 31/4196; A61K 31/575; A61K 31/635; A61K 45/06; A61P 19/02; A61P 29/00

(52) U.S. Cl. CPC A61K 31/4545; A61K 31/135; A61K 31/155; A61K 31/216; A61K 31/4196; A61K 31/575; A61K 31/635; A61K 45/06; A61P 19/02; A61P 29/00

(57) ABSTRACT

Compositions and methods are provided for inhibiting or treating the progression of inflammatory diseases by administration to an individual of an effective dose of a combination of active agents comprising or consisting essentially of a selective histamine H1 receptor antagonist; in combination with a second agent that provides for reduction of pain, inflammation, alteration of inflammatory lipids, and the like.

### CROSS REFERENCE TO RELATED APPLICATION

The present application is a 371 and claims the benefit of PCT Application No. PCT/US2021/057612, filed Nov. 1, 2021, which claims priority to U.S. Provisional Patent Application No. 63/108,072, filed Oct. 30, 2020, which applications are incorporated herein by reference in their entirety.

### STATEMENT REGARDING FEDERALLY SPONSORED RESEARCH

This invention was made with Government support under contract AR063676 awarded by the National Institutes of Health. The Government has certain rights in the invention.

### BACKGROUND OF THE INVENTION

Many diseases have an underlying inflammatory component that contributes to disease initiation and/or progression. Inflammatory diseases include degenerative diseases, such as osteoarthritis (OA), Alzheimer's disease (AD), and macular degeneration; autoimmune diseases, such rheumatoid arthritis (RA), juvenile rheumatoid arthritis (JRA), juvenile idiopathic arthritis (JIA), psoriatic arthritis (PSA), ankylosing spondylitis (AS) and multiple sclerosis (MS); chronic infections, such as infection with human immunodeficiency virus (HIV), chronic hepatitis C virus (HCV), chronic hepatitis B virus (HBV), chronic cytomegalovirus (CMV), mycobacterium tuberculosis (TB), or other chronic viral and bacterial infections; inflammatory metabolic diseases, such as type II diabetes and hepatic disease; cardiovascular diseases, such as atherosclerosis; cancers, which can arise from and induce inflammation; as well as pain in joint injury, dysmenorrhea and other conditions and diseases with an inflammatory component. In particular, the present invention relates to use of combination therapies described below as a composition and method for treating inflammatory diseases.

OA affects nearly 27 million people in the United States, accounting for 25% of visits to primary care physicians, and half of all prescriptions for non-steroidal anti-inflammatory drugs (NSAIDs). It is a chronic arthropathy characterized by disruption and potential loss of joint cartilage along with other joint changes, including bone remodeling such as bone hypertrophy (osteophyte formation), subchondral sclerosis, and formation of subchondral cysts. OA is viewed as failure of the synovial joint (Abramson et al, Arthritis Res Ther. 2009; 11(3): 227; Krasnokutsky et al, Osteoarthritis Cartilage. 2008;16 Suppl 3: S1-3; Brandt et al, Rheum Dis Clin North Am. 2008 August; 34(3): 531-59). OA results in the degradation of joints, including degradation of articular cartilage and subchondral bone, resulting in mechanical abnormalities and joint dysfunction. Symptoms may include joint pain, tenderness, stiffness, sometimes an effusion, and impaired joint function. A variety of causes can initiate processes leading to loss of cartilage in OA. A subgroup of OA patients exhibit a form of OA termed “erosive OA”, which includes erosive changes in the involved joints, typically involves the hands, and is clinically-distinct from the more common and typical form of OA that does not involve erosive changes (Punzi L, Best Pract Res Clin Rheumatol. 2004 18(5): 739-58); Belhorn L R, et. al. Semin Arthritis Rheum. 1993, 22 (5): 298-306). Although erosive OA has an inflammatory etiology, the studies described herein pertain to general non-erosive OA.

OA (the non-erosive and more common form) may begin with joint damage caused by trauma to the joint; mechanical injury to the meniscus, articular cartilage, a joint ligament, or other joint structure; defects in cartilage matrix components; and the like. Mechanical stress on joints may underlie the development of OA in many individuals, with the sources of such mechanical stress being many and varied, including misalignment of bones as a result of congenital or pathogenic causes; mechanical injury; overweight; loss of strength in muscles supporting joints; and impairment of peripheral nerves, leading to sudden or dyscoordinated movements that overstress joints.

Articular cartilage comprises chondrocytes that generate and are surrounded by extracellular matrix. In synovial joints there are at least two movable bony surfaces that are surrounded by the synovial membrane, which secretes synovial fluid, a transparent alkaline viscid fluid that fills the joint cavity, and articular cartilage, which is interposed between the articulating bony surfaces. The earliest gross pathologic finding in OA is softening of the articular cartilage in habitually loaded areas of the joint surface. This softening or swelling of the articular cartilage is frequently accompanied by loss of proteoglycans from the cartilage matrix. As OA progresses, the integrity of the cartilage surface is lost and the articular cartilage thins, with vertical clefts extending into the depth of the cartilage in a process called fibrillation. Joint motion may cause fibrillated cartilage to shed segments and thereby expose the bone underneath (subchondral bone). In OA, the subchondral bone is remodeled, featuring subchondral sclerosis, subchondral cycts, and ectopic bone comprising osteophytes. The osteophytes (bone spurs) form at the joint margins, and the subchondral cysts may be filled with synovial fluid. The remodeling of subchondral bone increases the mechanical strain and stresses on both the overlying articular cartilage and the subchondral bone, leading to further damage of both the cartilage and subchondral bone.

The tissue damage stimulates chondrocytes to attempt repair by increasing their production of proteoglycans and collagen. However, efforts at repair also stimulate the enzymes that degrade cartilage, as well as inflammatory cytokines, which are normally present in only small amounts. Inflammatory mediators trigger an inflammatory cycle that further stimulates the chondrocytes and synovial lining cells, eventually breaking down the cartilage. Chondrocytes undergo programmed cell death (apoptosis) in OA joints.

OA is characterized by low-grade infiltration of inflammatory cells, primarily macrophages, but also B cells and T cells. These cells, again primarily macrophages, are capable of producing inflammatory cytokines and matrix metalloproteases (MMPs) in the OA joint. However, when stimulated by inflammatory cytokines, such as IL-1 and TNF, tissue-resident cells within the joint, including synovial fibroblasts and chondrocytes, can produce additional inflammatory cytokines, including IL-6, as well as multiple MMPs.

OA should be suspected in patients with gradual onset of joint symptoms and signs, particularly in older adults, usually beginning with one or a few joints. Pain can be the earliest symptom, sometimes described as a deep ache. Pain is usually worsened by weight bearing and relieved by rest but can eventually become constant. Joint stiffness in OA is associated with awakening or inactivity. If OA is suspected, plain x-rays should be taken of the most symptomatic joints. X-rays generally reveal marginal osteophytes, narrowing of the joint space, increased density of the subchondral bone, subchondral cyst formation, bony remodeling, and joint effusions. Standing x-rays of knees are more sensitive in detecting joint-space narrowing. Magnetic resonance imaging (MRI) can be used to detect cartilage degeneration, and several MRI-based based scoring systems exist for characterizing the severity of OA (Hunter et al, PM R. 2012 May; 4(5 Suppl): S68-74).

OA commonly affects the hands, feet, spine, and the large weight-bearing joints, such as the hips and knees, although in theory any joint in the body can be affected. As OA progresses, the affected joints appear larger, are stiff and painful, and usually feel better with gentle use but worse with excessive or prolonged use. Treatment generally involves a combination of exercise, lifestyle modification, and analgesics. If pain becomes debilitating, joint-replacement surgery may be used to improve quality of life.

In addition to affecting humans, OA and joint degeneration also frequently impacts animals, including dogs, cats, horses, and other animals in which it can causes significant joint pain and dysfunction. Osteoarthritis (OA) is the most common form of arthritis in dogs, affecting approximately a quarter of the population. It is a chronic joint disease characterized by loss of joint cartilage, thickening of the joint capsule and new bone formation around the joint (osteophytosis) and ultimately leading to pain and limb dysfunction. The majority of OA in dogs occur secondarily to developmental orthopedic disease, such as cranial cruciate ligament disease, hip dysplasia, elbow dysplasia, OCD, patella (knee cap) dislocation. In a small subset of dogs, OA occurs with no obvious primary causes and can be related to genetic and age. Other contributing factors to OA in dogs include body weight, obesity, gender, exercise, and diet.

Among the agents proposed to modify disease in OA, such as doxycycline (presumably through its ability to inhibit MMPs), bisphosphonates (presumably through their ability to inhibit osteoclast activation), and licofelone (presumably through its ability to inhibit the cyclooxygenase and lipoxegenase pathways), none have been shown to afford robust chondroprotection as defined by slowing of cartilage breakdown. Among the agents that have demonstrated partial efficacy in controlling OA-associated pain are analgesics such as acetaminophen and anti-inflammatories such as NSAIDs, opiates, intra-articular corticosteroids, and hyaluronic acid derivatives injected into the joint. These agents have not been demonstrated to prevent cartilage loss or slow the loss of joint function. There is some evidence that mast cells are aberrantly activated in osteoarthritic joint tissues; and that the IgE/FcεRI/Syk signaling axis is involved in the development of osteoarthritis. Methods of disrupting the development of OA, preventing the progression of OA, and of treating the pain in OA is of interest for many clinical purposes, and is addressed herein.

Rheumatoid arthritis is a chronic syndrome characterized by usually symmetric inflammation of the peripheral joints, potentially resulting in progressive destruction of articular and periarticular structures, with or without generalized manifestations (Firestein (2003) Nature 423(6937): 356-61; McInnes and Schett. (2011) N Engl J Med. 365(23): 2205-19). About 0.6% of all populations are affected, women two to three times more often than men. Methods of preventing progression and treating pain in RA is of interest for many clinical purposes, and is addressed herein.

### SUMMARY OF THE INVENTION

Compositions and methods are provided for preventing or treating inflammatory diseases, by administration to an individual of an effective dose of a combination of active agents comprising or consisting essentially of a selective histamine H1 receptor antagonist; in combination with a second agent that provides for reduction of pain, inflammation, lipids, etc., as defined herein. In some embodiments the inflammatory disease is osteoarthritis (OA). In some embodiments the selective histamine H1 receptor antagonist is rupatadine. In some embodiments, pain associated with osteoarthritis is reduced with the combination therapy provided herein. In some embodiments, disease progression of osteoarthritis is reduced with the combination therapy provided herein. In some embodiments the combination provides for a synergistic benefit, relative to treatment with a single agent, for reducing disease progression and/or reducing pain.

Treatment of inflammatory disease by the methods of the invention can substantially reduce or prevent disease progression and development of clinical symptoms. In some embodiments, treatment is initiated at a “pre-clinical” time point, as defined herein. In some embodiments, treatment is initiated for established disease, wherein pain associated with the condition is reduced relative to an untreated or control individual; and/or wherein disease progression is reduced relative to an intreated or control individual. Administration of the combination therapy of the invention may continue for an extended period of time, for example over a period of months or years.

The active agents can be administered separately, or can be co-formulated in a single-unit dose. Each or both of the active agents can be formulated in various ways, including without limitation a solid oral dosage form, for example in a unit dose pill, capsule, etc. An oral dosage form may provide for delayed-release or sustained-release in a controlled manner over at least a 12-hour period, a 24-hour period, etc.

In some embodiments the selective histamine H1 receptor antagonist also blocks receptors of platelet-activating factor (PAF). In some embodiments a histamine antagonist with H1 receptor antagonist activity is rupatadine. In some embodiments the selective H1 receptor is cetirizine. In some embodiments, a drug combination pharmaceutical formulation comprising an effective dose of a selective histamine H1 receptor antagonist, e.g. rupatadine, and a pharmaceutically acceptable excipient is provided. The drug combination may comprise or consist of a combination of the selective H1 receptor antagonist with a COX-2 inhibitor, e.g. celecoxib; in combination with a fibrate, e.g. fenofibrate; in combination with another PPARα antagonist, e.g. TPST-1120; in combination with an SSRI, e.g. sertraline; in combination with a second selective antagonist of histamine H1 receptor, e.g. cetirizine; in combination with metformin; in combination with a bile acid; etc. In some embodiments, a formulation comprises a unit dose of the combination therapy.

In some embodiments, the formulation comprises or consists essentially of a selective histamine H1 receptor antagonist, e.g. rupatadine, and an effective dose of celecoxib. In some embodiments, the formulation comprises or consists essentially of a selective histamine H1 receptor antagonist, e.g. rupatadine, and an effective dose of fenofibrate. In some embodiments, the formulation comprises or consists essentially of a selective histamine H1 receptor antagonist, e.g. rupatadine, and an effective dose of sertraline. In some embodiments, the formulation comprises or consists essentially of a selective histamine H1 receptor antagonist, e.g. rupatadine, and an effective dose of cetirizine.

In some embodiments, the combination of active agents comprises or consists essentially of rupatadine as the selective histamine H1 receptor agonist, or an equivalent, in a daily fixed dose of at least about 2.5 mg, at least about 5 mg, at least about 10 mg, at least about 20 mg, at least about 30 mg, at least about 40 mg, and not more than about 100 mg, not more than about 75 mg, not more than about 50 mg. The dose may be, for example, from 2.5 to 5 mg/day, from 5 to 10 mg/day, from 5 to 20 mg/day, from 5 to 40 mg/day; from 10-30 mg/day, from 10-20 mg/day, from 20-40 mg/day, etc.

In some embodiments, treatment of an individual with the methods disclosed herein decreases pain in the individual. In some embodiments, treatment of an individual with the methods disclosed herein decreases or stabilizes indicia of inflammation in the individual. In some embodiments, treatment of an individual with the methods disclosed herein decreases or stabilizes cartilage degeneration. Efficacy of therapy can be determined at a suitable timepoint after initiation of the treatment, for example after at least about 4 weeks, after at least about 8 weeks, after at least about 12 weeks, after at least about 3 months, after at least about 4 months, after at least about 5 months, after at least about 6 months, or more, e.g. up to about 9 months, up to about 1 year, up to about 2 years, etc.

In some embodiments a package suitable for use in commerce is provided for treating inflammation according to the methods of the invention, e.g. a pharmaceutical formulation comprising or consisting essentially of a selective histamine H1 receptor antagonist, e.g. rupatadine, in combination with a second agent, e.g. example in combination of a COX-2 inhibitor; in combination with a fibrate, e.g. fenofibrate; in combination with a PPARα antagonist, e.g. TPST-1120; in combination with an SSRI, e.g. sertraline; in combination with a second selective antagonist of histamine H1 receptor, e.g. cetirizine; etc.; and associated with the package, printed instructional and informational material, which may be attached to the package, or displayed as an integral part of the package, said instructional and informational material stating in words which convey to a reader thereof that the active ingredients, when administered to an individual for treatment of inflammatory disease such as osteoarthritis, will ameliorate, reduce pain, diminish, actively treat, reverse or prevent any injury, damage or loss of tissue. The package as above-described may conform to all regulatory requirements relating to the sale and use of drugs, including especially instructional and informational material.

In some embodiments the methods of the disclosure comprise the step of determining the presence of early-stage arthritis in an individual or susceptibility to development of arthritis prior to treatment, and thus a need of treatment. The method may further include determining the presence of inflammation, prior to the administering step, where an individual in an early stage of arthritis showing signs of inflammation, particularly inflammation of arthritic joints, is selected for treatment with the combination therapy of the invention. In some embodiments the treatment or prevention ameliorates, diminishes, actively treats, reverses or prevents injury, damage, or loss of articular cartilage or subchondral bone subsequent to the early stage of disease. In some embodiments the arthritis is OA.

The determination of early-stage arthritis or a pre-arthritis condition in an individual can comprise analyzing the individual for the presence of at least one marker indicative of the presence of early or pre-arthritis. In some embodiments the method comprises analyzing an individual for the presence of one, two, three, four or more markers that are diagnostic for early or pre-arthritis. In some embodiments at least one of the marker(s) is an imaging marker, including without limitation: arthroscopy, radiographic imaging, ultrasound imaging, magnetic resonance imaging (MRI), computed tomography (CT), etc. In some embodiments at least one of the marker(s) is a molecular marker or a marker of inflammation, where a biological sample is obtained from the individual and analyzed for the presence of a molecule, e.g. C-reactive protein (CRP), a cytokine, antibody, cartilage component, protease, etc. or other clinical laboratory marker of inflammation, e.g. erythrocyte sedimentation rate (ESR), and compared to a control or reference value, wherein altered level of the molecular marker, alone or in combination with the imaging marker, is indicative of early arthritis.

### BRIEF DESCRIPTION OF THE DRAWINGS

FIGS. 1A-1B Rupatadine plus celecoxib small molecule combination reduced pain and inflammatory mediator expression in human OA synovial tissue organoid stimulation assays (**p<0.01). A. Expression of IL-1β. B. Expression of MMP13.

FIG. 2. Rupatadine plus celecoxib small molecule combination synergistically reduced pain in the mouse DMM model for OA (**p<0.01).

FIG. 3. Rupatadine plus celecoxib small molecule combination synergistically reduced cartilage degeneration in the DMM mouse model for OA (*p<0.05, **p<0.01, ***p<0.001, N.S. non-significant; symbols directly above bars represent comparisons to the vehicle-treated group).

FIG. 4. Cetirizine plus celecoxib small molecule combination synergistically reduced cartilage degeneration in the DMM mouse model for OA (*p<0.05, **p<0.01, ***p<0.001; symbols directly above bars represent comparisons to the vehicle-treated group).

FIG. 5. Rupatadine plus celecoxib small molecule combination synergistically reduced human OA synoviocyte expression of inflammatory and degradative mediators. # indicates synergy.

FIG. 6. Cetirizine plus celecoxib small molecule combination synergistically reduced human OA synoviocyte expression of inflammatory and degradative mediators. # indicates synergy.

FIG. 7. Rupatadine plus celecoxib small molecule combination synergistically reduced human OA chondrocyte expression of degradative mediators. # indicates synergy.

FIG. 8. Cetirizine plus celecoxib small molecule combination synergistically reduced human OA chondrocyte expression of degradative mediators. # indicates synergy.

FIG. 9. Rupatadine plus fenofibrate small molecule combination reduced inflammatory lipid PAF biosynthesis enzyme (Ipcat2) gene expression as well as PAF receptor (ptafr) gene expression in human OA synovial tissue organoid stimulation assays (*p<0.05, **p<0.01, ***p<0.001, N.S. non-significant; symbols directly above bars represent comparisons to the vehicle-treated group).

FIGS. 10A-10B. Rupatadine plus fenofibrate small molecule combination reduced pain and inflammatory mediator expression in human OA synovial tissue organoid stimulation assays (**p<0.01; symbols directly above bars represent comparisons to the vehicle-treated group). A. IL-1β expression. B. MMP13 expression.

FIGS. 11A-11B. Rupatadine plus fenofibrate drug combination prevented development of OA in the DMM mouse model. A. Cartilage degeneration scores. B. Representative safranin-O-stained joint tissue sections. (*p<0.05, **p<0.01, N.S. non-significant; symbols directly above bars represent comparisons to the vehicle-treated group).

FIG. 12. Clinical trial design.

FIG. 13. Normal and pathogenic lipid metabolism pathways. Left: Cellular metabolism of LDL in healthy joints. Inflammatory lipid precursors including LDLs (comprised of lipids and apolipoproteins such as ApoE) are transported into the cell upon binding cognate receptors. Caveolin-1 promotes formation of caveolae in the plasma membrane upon binding LDLs. Following uptake, LDLs are trafficked to the ER and processed. PPARα: (i) activates lipid metabolism and thereby reduces inflammatory lipid generation, and (ii) inhibits NF-□B activation. Right: Pathogenic cellular metabolism of modified LDLs and plasmalogens in OA. Inflammatory lipids result from modification of extracellular LDL and phospholipids to oxLDL, acLDL and plasmalogens. Uptake of oxLDL and acLDL, mediated by CD36, TLR2, and TLR4 and uptake of plasmalogens promote activation of NFκB and downstream expression of inflammatory mediators. Rupatadine is a PAFR (platelet activating factor inflammatory lipid receptor antagonist). Fenofibrate+rupatadine combination treatment synergistically reduces LPCAT2 expression, thereby reducing PAF production.

FIG. 14. Atorvastatin does not prevent OA in mice. C57BL/6J (wild-type) mice were treated with atorvastatin (0.5 mg/kg) or vehicle for 12 wks following DMM. Quantification of cartilage degeneration 20 wks post-DMM; p=0.0513.

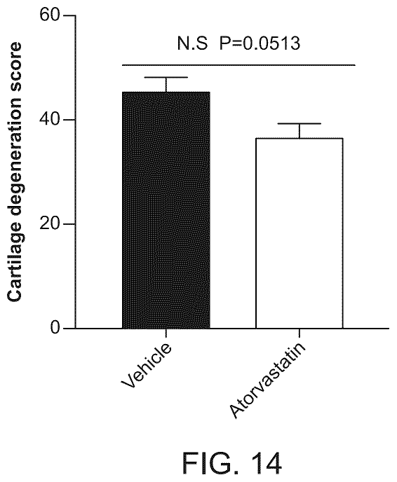

### DETAILED DESCRIPTION OF THE EMBODIMENTS

Compositions and methods are provided for preventing or treating the early stages of inflammatory diseases, including autoimmune diseases, degenerative inflammatory diseases, metabolic inflammatory diseases, cancer associated with inflammation, and other inflammatory diseases by administration to an individual of an effective dose of a combination of a combination therapy comprising a selective histamine H1 receptor antagonist, e.g. rupatadine, in combination with a second agent. In some embodiments the compositions are utilized to treat osteoarthritis, with the purpose of preventing any of the following: cartilage destruction, pain, and/or loss of joint function; and/or for the reduction of pain associated with OA.

All publications, patents, and patent applications mentioned in this specification are herein incorporated by reference to the same extent as if each individual publication, patent, or patent application was specifically and individually indicated to be incorporated by reference.

While preferred aspects of the present disclosure have been shown and described herein, it is to be understood that the disclosure is not limited to the particular aspects of the disclosure described below, as variations of the particular aspects may be made and still fall within the scope of the appended claims. It is also to be understood that the terminology employed is for the purpose of describing particular aspects of the disclosure, and is not intended to be limiting. Instead, the scope of the present disclosure is established by the appended claims. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the disclosure provided herein.

Unless defined otherwise, all technical and scientific terms used herein have the same meaning as commonly understood to one of ordinary skill in the art to which this disclosure belongs. Although any methods, devices and materials similar or equivalent to those described herein can be used in the practice or testing of the disclosure, the preferred methods, devices and materials are now described.

Inflammatory disorder. Inflammatory disorders are conditions that involve inflammation. The presence of inflammation can be detected by a variety of approaches, including clinical history, physical examination, laboratory testing, histologic analysis of tissue, analysis of biomarkers, and imaging. Clinical features and physical exam features of inflammation include swelling, effusions, edema, redness, warmth, pain, or associated pathologically with the influx of inflammatory cells or production of inflammatory mediators.

Low-grade inflammation. The presence of low-grade inflammation is characterized by a elevations in the local or systemic concentrations of cytokines such as TNF-α, IL-6, and c-reactive protein (CRP), and occurs in adiposity, osteoarthritis, Alzheimer's disease, metabolic syndrome, and many chronic and degenerative diseases. Low-grade inflammation is manifest by inflammation present at a level below the “high-grade” inflammation detected in active autoimmune diseases (such as active rheumatoid arthritis, psoriasis, Crohn's disease, systemic lupus erythematosus and other autoimmune states) and in certain viral and bacterial infections during which humans experience clinical symptoms (such as influenza virus infection, Staphylococcus aureus infection, and other infections).

Amelioration of an inflammatory disorder. The reduction of inflammation as indicated by dissipation of inflammation, a reduction in number of inflammatory cells or in levels of inflammatory mediators as evidenced by symptomatic relief (including but not limited to pain relief), radiographic changes, biochemical changes, pathologic/histologic changes, or decreased progression of such markers of inflammation or disease.

Cytokines as mediators of pain. Cytokines and chemokines are recognized as important mediators of inflammatory and neuropathic pain, supporting system sensitization and the development of a persistent pathologic pain (Cook et al, Trends in Immunology, 2018, 39(3): 240-255; Miller et al, Cytokin, 2018, 39(3): 185-193; White et al, Curr Opin Anaesthesiol. 2008October; 21(5): 580-585). Cytokines (including chemokines) can induce a facilitation of nociceptive processing at all levels of the neuraxis including supraspinal centers where nociceptive input evokes an affective component of the pain state. Multiple proinflammatory and anti-inflammatory cytokines/chemokines contribute to pain at multiple levels of neuronal organization: (1) peripheral nociceptor termini; (2) dorsal root ganglia; (3) spinal cord; and (4) supraspinal areas. Thus, current thinking suggests that cytokines by this action throughout the neuraxis play key roles in the induction of pain and the maintenance of the facilitated states of pain behavior generated by tissue injury/inflammation and nerve injury. It is believed that cytokines and chemokines contribute to pain in OA, RA, and other inflammatory diseases of the joints and other tissues. Immune cytokines and their cognate receptors that contribute to pain include TNF, interleukin-1b (IL-1b), IL-6, IL-10, IL-15, MCP-1/CCL2, SDF-1/CXCL12, CCR2, driving both local tissue damage and pain in arthritis and inflammatory diseases affecting other tissues. Measurement of these pain-mediating and pain-associated cytokines can provide a surrogate measure for mediators of pain, and reduction of these cytokines has been associated with reductions in pain.

Administration of agents. Administration of a drug or other chemical entity to an animal, human or other mammal via any route including but not limited to oral, intradermal, intramuscular, intraperitoneal, or intravenous.

Pharmaceutical formulation. The process by which different chemical substances including but not limited to active drugs are combined and formulated for the treatment of humans. Sterile formulation. A formulation free of living germs or microorganisms.

Therapeutically effective amount. The mass of active drug in and frequency of administration of a formulation that results in the prevention of the development of a disease, prevention of the progression of a disease, reduction in the severity of a disease, or treatment of disease symptoms as defined above.

Dose range for each individual agent. The range of the mass of active drug in and frequency of administration of a formulation which results in the prevention of the development of a disease, prevention of the progression of a disease, reduction in the severity of a disease, or treatment of disease symptoms as defined above.

Regimen. Regimen means dose, frequency of administration, for example twice-per day, daily, weekly, bi-weekly etc., and duration of treatment, for example one day, several days, one week, several weeks, one month, several months, one year, several years, etc.

Loading dose. A large initial dose of a substance or series of such doses given to more rapidly achieve a therapeutic concentration in the body. A loading dose can be higher or lower than the maintenance dose. In some instances, therapy is initiated at a loading dose for days, weeks or months in order to rapidly achieve therapeutic levels of the drug or other chemical entity in tissue, then the dose is lowered to the long-term maintenance dose.

Dose pack. A premeasured amount of drug to be dispensed to a patient in a set or variable dose and in a package including but not limited to a blister pack or other series of container for the purpose of facilitating a dose regimen. A dose pack can be used to facilitate delivery of an initial and/or loading dose to an individual, followed by a maintenance dose.

Biomarker (also referred to herein as a “marker”). A biomarker is an objectively measured characteristic that reflects a biological condition, pre-disease state, or disease state including but not limited to molecular, biochemical, imaging, or gross physical measurements.

Imaging biomarker (also referred to herein as an “imaging marker”). A biomarker that is measured or otherwise determined through use of an imaging modality, including but not limited to ultrasound, radiography, computerized tomography, magnetic resonance imaging, or nuclear medical scanning.

Biochemical biomarker (also referred to herein as a “biochemical marker”). A biologic substance that is measured in blood, urine or other tissue as a biomarker. Biological biomarkers of interest include without limitation proteins, nucleic acids, metabolites, fatty acids, inflammatory lipids as taught herein, peptides, and the like. In one embodiment the biochemical marker is a biomarker of inflammation. In one embodiment the biomarker is C reactive protein (CRP) detected in blood. In another embodiment the biochemical biomarker is collagen type II (CTX-II) C-telopeptide degradation products that can be detected in urine or blood.

Biomarker of inflammation (also referred to as an “inflammatory marker”). A biomarker of inflammation can include cytokines, inflammatory lipids, and other laboratory markers of inflammation, including C reactive protein (CRP) and the erythrocyte sedimentation rate (ESR).

Reference range is defined as the set of values within which 95 percent of the normal population falls. It typically refers to the value of a biomarker, and examples of such biomarkers include but are not limited imaging biomarkers, biochemical biomarkers, clinical biomarker, radiographic biomarkers, and other biomarkers.

The phrase “pharmaceutically acceptable salt(s)”, as used herein, means those salts of compounds of the invention that are safe and effective for oral and topical use in mammals and that possess the desired biological activity. Pharmaceutically acceptable salts include salts of acidic or basic groups present in compounds of the invention. Pharmaceutically acceptable salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, pamoate (i.e., 1,1′-methylene-bis-(2-hydroxy-3-naphthoate)), aluminum, calcium, lithium, magnesium, potassium, sodium, zinc, and diethanolamine salts and the like, as known in the art.

Selective histamine H1 receptor antagonists. Histamine is a biologically active substance that potentiates the inflammatory and immune responses of the body, regulates physiological function in the gut, and acts as a neurotransmitter. Histamine antagonists are drugs that antagonize these effects by blocking or inhibiting histamine receptors. They are categorized as either H1 or H2 according to the type of H receptor targeted. H1 antihistamines are mostly used to treat allergic reactions and mast cell-mediated disorders. This subtype is further divided into two generations. While first-generation H1 antihistamines have a central effect and, thus, are also used as sedatives, second-generation H1 antihistamines are selective for peripheral effects.

Selective peripheral H1 receptor antagonists are generally available as oral formulations and have known dosing information. For example, Fexofenadine (Allegra); Cetirizine (Zyrtec); Loratadine (Claritin); Rupatadine (Rupafin); Astemizole (Hismanal); Ketotifen (Zaditor); Mizolastine (Mizollen); Acrivastine (Benadryl Allergy Relief (UK), Semprex (US)); Ebastine (Evastin, Kestine, Ebastel, Aleva, Ebatrol); Bilastine (Bilaxten); Bepotastine (Talion, Bepreve); Terfenadine (Seldane (US), Triludan (UK), and Teldane (Australia)); Quifenadine (Phencarol), etc. are known in the art and can be used in the methods disclosed herein.

Rupatadine is a second generation antihistamine and platelet activating factor (PAF) antagonist used to treat allergies. Rupatadine fumarate has been approved for the treatment of allergic rhinitis and chronic urticaria in adults and children over 12 years. It is available as round, light salmon colored tablets containing 10 mg of rupatadine (as fumarate) to be administered orally, once a day. The efficacy of rupatadine as treatment for allergic rhinitis (AR) and chronic idiopathic urticaria (CIU) has been investigated in adults and adolescents (aged over 12 years) in several controlled studies, showing a rapid onset of action and a good safety profile even in prolonged treatment periods of a year. Rupatadine is a second generation, non-sedating, long-acting histamine antagonist with selective peripheral H1 receptor antagonist activity. It further blocks the receptors of the platelet-activating factor (PAF) according to in vitro and in vivo studies. Rupatadine possesses anti-allergic properties, such as the inhibition of the degranulation of mast cells induced by immunological and non-immunological stimuli, and inhibition of the release of cytokines, particularly of the tumor necrosis factors (TNF) in human mast cells and monocytes. In some embodiments the rupatadine dose for the methods disclosed herein is around 2.5 mg, or around 5 mg, or 7.5 mg, or 10 mg, or 12.5 mg

In some embodiments a histamine antagonist with selective peripheral H1 receptor antagonist activity further blocks receptors of platelet-activating factor (PAF). In some preferred embodiments the histamine antagonist with selective peripheral H1 receptor antagonist activity is rupatadine.

Fibrates. Fibric acid derivatives (fibrates) are a class of medication that lowers blood triglyceride levels. Fibrates lower blood triglyceride levels by reducing the liver's production of VLDL (the triglyceride-carrying particle that circulates in the blood) and by speeding up the removal of triglycerides from the blood.

Fibrates activate peroxisome proliferator activated receptor (PPAR) alpha, which controls the expression of gene products that mediate the metabolism of TG and HDL. As a result, synthesis of fatty acids, TG and VLDL is reduced, while that of lipoprotein lipase, which catabolises TG, is enhanced. In addition, production of Apo A1 and ATP binding cassette A1 is up-regulated, leading to increased reverse cholesterol transport via HDL. Fibrates are structurally and pharmacologically related to the thiazolidinediones, a novel class of anti-diabetic drugs that also act on PPARs (more specifically PPARγ). Examples of fibrates include, without limitation, Aluminium clofibrate, Bezafibrate, Ciprofibrate, Choline fenofibrate, Clinofibrate, Clofibrate, Clofibride, Fenofibrate, Gemfibrozil, Ronifibrate, Simfibrate. In some embodiments the fibrate for use in the combination therapy disclosed herein is fenofibrate.

Fenofibrate is mainly used for primary hypercholesterolemia or mixed dyslipidemia. Fenofibrate appears to decrease the risk of cardiovascular disease and possibly diabetic retinopathy in those with diabetes mellitus, and firstly indicated for the reduction in the progression of diabetic retinopathy in patients with type 2 diabetes. Fenofibrate also has an use as an added therapy of high blood uric acid levels in people who have gout. It is used in addition to diet to reduce elevated low-density lipoprotein cholesterol (LDL), total cholesterol, triglycerides (TG), and apolipoprotein B (apo B), and to increase high-density lipoprotein cholesterol (HDL) in adults with primary hypercholesterolemia or mixed dyslipidemia. Fenofibrate can be dosed as 54, 67, 145, and 160 mg tablets, as well as 67, 134, and 200 mg micronized capsules. The differences among strengths are a result of altered bioavailability (the fraction absorbed by the body) due to particle size. For example, 200 mg can be replaced by 160 mg micronized fenofibrate. The 145 mg strength that appeared in 2005-2006 is nanonised (i.e. the particle size is below 400 nm).

For the purposes of the present invention, an effective dose of fenofibrate or another fibrate in a combination with rupatadine is the dose that, when administered for a suitable period of time, usually at least about one week, and may be about two weeks, or more, up to extended periods of time of months or years, will reduce the progression of the disease. It will be understood by those of skill in the art that an initial dose may be administered for such periods of time, followed by maintenance doses, which, in some cases, will be at a reduced dosage.

In some embodiments, for the methods disclosed herein, the dose of fenofibrate is between 25 to 400 mg/day, or between 50 to 200 mg/day, in a regular, micronized, or nanonised form. The dose of fenofibrate can be about 25 mg/day, or about 50 mg/day, or about 75 mg/day, or about 100 mg/day, or about 125 mg/day, or about 150 mg/day, or about 175 mg/day, or about 200 mg/day, or about 225 mg/day, or about 250 mg/day, in a regular, micronized, or nanonised form. The dose of rupatadine is generally 2.5-20 mg/day, and can be about 2.5 mg/day, or about 5 mg/day, or about 7.5 mg/day, or about 10 mg/day, or about 12.5 mg/day, or about 15 mg/day, or about 17.5 mg/day, or about 20 mg/day. In some embodiments a co-formulated combination is used for once-daily or twice-daily oral dosing is used that includes fenofibrate 145 mg+rupatadine 5 mg; or fenofibrate 145 mg+rupatadine 10 mg; or fenofibrate 145 mg+rupatadine 15 mg; or fenofibrate 145 mg+rupatadine 20 mg; or fenofibrate 100 mg+rupatadine 5 mg; or fenofibrate 100 mg+rupatadine 10 mg; or fenofibrate 100+rupatadine 15 mg; or fenofibrate 100+rupatadine 20 mg; or fenofibrate 200 mg+rupatadine 5 mg; or fenofibrate 200 mg+rupatadine 10 mg; or fenofibrate 200+rupatadine 15 mg; or fenofibrate 200+rupatadine 20 mg.

The formulation and administration of fibrates is well known, and will generally follow conventional usage. The dosage required to treat inflammatory disease may be commensurate with the dose used in treating hypercholesterolemia.

In addition to fibrates, other PPARα antagonists can be used. An example of such a small molecule, selective and competitive antagonist of peroxisome proliferator activated receptor alpha (PPARα) is TPST-1120. Upon oral administration, TPST-1120 targets, binds to and blocks the activity of PPARα, thereby blocking transcription of PPARα target genes leading to an intracellular metabolism shift from fatty acid oxidation (FAO) to glycolysis in FAO-dependent tumors and reducing the production of fatty acids in the tumor microenvironment (TME). As fatty acids are essential for tumor cell growth in FAO-dependent tumor cells and are needed for the metabolism of suppressive immune cells in the TME, including regulatory T-cells (Tregs), reducing the amount of fatty acids leads to a direct killing of FAO-dependent tumor cells. It also skews macrophages from the immune suppressive M2 phenotype to an effector M1 phenotype and facilitates the cytotoxicity of immune effector cells, thereby stimulating an anti-tumor immune response and further killing tumor cells. TPST-1120 also restores the natural inhibitor of angiogenesis thrombospondin-1 (TSP-1) and stimulator of interferon genes (STING) in the TME. PPARα, a ligand-activated nuclear transcription factor and metabolic checkpoint, regulates the expression of FAO genes and lipid metabolism. It plays a key role in immunosuppression in the TME.

For the purposes of the present invention, an effective dose of TPST-1120 in a combination with rupatadine is the dose that, when administered for a suitable period of time, usually at least about one week, and may be about two weeks, or more, up to extended periods of time of months or years, will reduce the progression of the disease. It will be understood by those of skill in the art that an initial dose may be administered for such periods of time, followed by maintenance doses, which, in some cases, will be at a reduced dosage. The formulation and administration TPST-1120 may generally follow conventional usage.

COX-2 inhibitors are a type of nonsteroidal anti-inflammatory drug (NSAID) that directly targets cyclooxygenase-2, COX-2, an enzyme responsible for inflammation and pain. Targeting selectivity for COX-2 reduces the risk of peptic ulceration and is the main feature of celecoxib, rofecoxib, and other members of this drug class. Examples of COX-2 inhibitors include, without limitation, etoricoxib, rofecoxib, celecoxib, 2,5-dimethyl-celecoxib, valdecoxib, meloxicam, etc. In some embodiments a COX-2 inhibitor for use in the combination therapy disclosed herein is celecoxib.

Celecoxib is a highly selective reversible inhibitor of the COX-2 isoform of cyclooxygenase, celecoxib inhibits the transformation of arachidonic acid to prostaglandin precursors. It is used to treat the pain and inflammation in osteoarthritis, acute pain in adults, rheumatoid arthritis, ankylosing spondylitis, painful menstruation, and juvenile rheumatoid arthritis. It may also be used to decrease the risk of colorectal adenomas in people with familial adenomatous polyposis.

For the purposes of the present invention, an effective dose of celecoxib or another COX-2 inhibitor in a combination with rupatadine is the dose that, when administered for a suitable period of time, usually at least about one week, and may be about two weeks, or more, up to extended periods of time of months or years, will reduce the progression of the disease and/or pain associated with the disease. It will be understood by those of skill in the art that an initial dose may be administered for such periods of time, followed by maintenance doses, which, in some cases, will be at a reduced dosage.

The formulation and administration of celecoxib and other COX-2 inhibitors is well known, and will generally follow conventional usage. The dosage required to treat inflammatory disease may be commensurate with the dose used in reducing pain. In some embodiments the dose of celecoxib for the methods disclosed herein is about 25 mg/day, or about 50 mg/day, or about 75 mg/day, or about 100 mg/day, or about 125 mg/day, or about 150 mg/day, or about 175 mg/day, or about 200 mg/day, or about 250 mg/day, or about 300 mg/day, or about 350 mg/day, or about 400 mg/day, or about 450 mg/day, or about 500 mg/day. In some embodiments the dose of celecoxib can be between 25-600 mg/day, or between 50-200 mg/day, or between 50-100 mg/day. The dose of rupatadine is generally 2.5-20 mg/day, and can be about 2.5 mg/day, or about 5 mg/day, or about 7.5 mg/day or about 10 mg/day, or about 12.5 mg/day, or about 15 mg/day, or about 17.5 mg/day, or about 20 mg/day, or about 22.5 mg/day, or about 25 mg/day, or about 30 mg/day. In some embodiments a co-formulated combination is used for once-daily or twice-daily oral dosing is used that includes celecoxib 100 mg+rupatadine 2.5 mg; 100 mg+rupatadine 5 mg; or celecoxib 100 mg+rupatadine 7.5 mg; or celecoxib 100 mg+rupatadine 10 mg; or celecoxib 100 mg+rupatadine 12.5 mg; or celecoxib 25 mg+rupatadine 2.5 mg; or celecoxib 25 mg+rupatadine 5 mg, or celecoxib 25 mg+rupatadine 7.5 mg; or celecoxib 25 mg+rupatadine 10 mg; or celecoxib 25 mg+rupatadine 12.5 mg, or celecoxib 25 mg+rupatadine 15 mg; or celecoxib 25 mg+rupatadine 17.5 mg; or celecoxib 25 mg+rupatadine 20 mg; or celecoxib 50 mg+rupatadine 2.5 mg; or celecoxib 50 mg+rupatadine 5 mg, or celecoxib 50 mg+rupatadine 7.5 mg; or celecoxib 50 mg+rupatadine 10 mg; or celecoxib 50 mg+rupatadine 12.5 mg, or celecoxib 50 mg+rupatadine 15 mg; or celecoxib 50 mg+rupatadine 17.5 mg; or celecoxib 50 mg+rupatadine 20 mg; or celecoxib 75 mg+rupatadine 2.5 mg; or celecoxib 75 mg+rupatadine 5 mg, or celecoxib 75 mg+rupatadine 7.5 mg; or celecoxib 75 mg+rupatadine 10 mg; or celecoxib 75 mg+rupatadine 12.5 mg, or celecoxib 75 mg+rupatadine 15 mg; or celecoxib 75 mg+rupatadine 17.5 mg; or celecoxib 50 mg+rupatadine 20 mg; or celecoxib 125 mg+rupatadine 2.5 mg; or celecoxib 125 mg+rupatadine 5 mg, or celecoxib 125 mg+rupatadine 7.5 mg; or celecoxib 125 mg+rupatadine 10 mg; or celecoxib 125 mg+rupatadine 12.5 mg, or celecoxib 125 mg+rupatadine 15 mg; or celecoxib 125 mg+rupatadine 17.5 mg; or celecoxib 125 mg+rupatadine 20 mg; or celecoxib 150 mg+rupatadine 2.5 mg; or celecoxib 150 mg+rupatadine 5 mg, or celecoxib 150 mg+rupatadine 7.5 mg; or celecoxib 150 mg+rupatadine 10 mg; or celecoxib 150 mg+rupatadine 12.5 mg, or celecoxib 150 mg+rupatadine 15 mg; or celecoxib 150 mg+rupatadine 17.5 mg; or celecoxib 150 mg+rupatadine 20 mg; or celecoxib 175 mg+rupatadine 2.5 mg; or celecoxib 175 mg+rupatadine 5 mg, or celecoxib 175 mg+rupatadine 7.5 mg; or celecoxib 175 mg+rupatadine 10 mg; or celecoxib 175 mg+rupatadine 12.5 mg, or celecoxib 175 mg+rupatadine 15 mg; or celecoxib 175 mg+rupatadine 17.5 mg; or celecoxib 175 mg+rupatadine 20 mg; or celecoxib 200 mg+rupatadine 2.5 mg; or celecoxib 200 mg+rupatadine 5 mg, or celecoxib 200 mg+rupatadine 7.5 mg; or celecoxib 200 mg+rupatadine 10 mg; or celecoxib 200 mg+rupatadine 12.5 mg, or celecoxib 200 mg+rupatadine 15 mg; or celecoxib 200 mg+rupatadine 17.5 mg; or celecoxib 200 mg+rupatadine 20 mg; or celecoxib 225 mg+rupatadine 2.5 mg; or celecoxib 225 mg+rupatadine 5 mg, or celecoxib 225 mg+rupatadine 7.5 mg; or celecoxib 225 mg+rupatadine 10 mg; or celecoxib 225 mg+rupatadine 12.5 mg, or celecoxib 225 mg+rupatadine 15 mg; or celecoxib 225 mg+rupatadine 17.5 mg; or celecoxib 225 mg+rupatadine 20 mg; or celecoxib 250 mg+rupatadine 2.5 mg; or celecoxib 250 mg+rupatadine 5 mg, or celecoxib 250 mg+rupatadine 7.5 mg; or celecoxib 250 mg+rupatadine 10 mg; or celecoxib 250 mg+rupatadine 12.5 mg, or celecoxib 250 mg+rupatadine 15 mg; or celecoxib 250 mg+rupatadine 17.5 mg; or celecoxib 250 mg+rupatadine 20 mg; or celecoxib 300 mg+rupatadine 2.5 mg; or celecoxib 300 mg+rupatadine 5 mg, or celecoxib 300 mg+rupatadine 7.5 mg; or celecoxib 300 mg+rupatadine 10 mg; or celecoxib 300 mg+rupatadine 12.5 mg, or celecoxib 300 mg+rupatadine 15 mg; or celecoxib 300 mg+rupatadine 17.5 mg; or celecoxib 125 mg+rupatadine 20 mg; or celecoxib 350 mg+rupatadine 2.5 mg; or celecoxib 350 mg+rupatadine 5 mg, or celecoxib 350 mg+rupatadine 7.5 mg; or celecoxib 350 mg+rupatadine 10 mg; or celecoxib 350 mg+rupatadine 12.5 mg, or celecoxib 350 mg+rupatadine 15 mg; or celecoxib 350 mg+rupatadine 17.5 mg; or celecoxib 350 mg+rupatadine 20 mg; or celecoxib 400 mg+rupatadine 2.5 mg; or celecoxib 400 mg+rupatadine 5 mg, or celecoxib 400 mg+rupatadine 7.5 mg; or celecoxib 400 mg+rupatadine 10 mg; or celecoxib 400 mg+rupatadine 12.5 mg, or celecoxib 400 mg+rupatadine 15 mg; or celecoxib 400 mg+rupatadine 17.5 mg; or celecoxib 400 mg+rupatadine 20 mg; or celecoxib 450 mg+rupatadine 2.5 mg; or celecoxib 450 mg+rupatadine 5 mg, or celecoxib 450 mg+rupatadine 7.5 mg; or celecoxib 450 mg+rupatadine 10 mg; or celecoxib 450 mg+rupatadine 12.5 mg, or celecoxib 450 mg+rupatadine 15 mg; or celecoxib 450 mg+rupatadine 17.5 mg; or celecoxib 450 mg+rupatadine 20 mg; or celecoxib 500 mg+rupatadine 2.5 mg; or celecoxib 500 mg+rupatadine 5 mg, or celecoxib 500 mg+rupatadine 7.5 mg; or celecoxib 500 mg+rupatadine 10 mg; or celecoxib 500 mg+rupatadine 12.5 mg, or celecoxib 500 mg+rupatadine 15 mg; or celecoxib 500 mg+rupatadine 17.5 mg; or celecoxib 500 mg+rupatadine 20 mg; or celecoxib 550 mg+rupatadine 2.5 mg; or celecoxib 550 mg+rupatadine 5 mg, or celecoxib 550 mg+rupatadine 7.5 mg; or celecoxib 550 mg+rupatadine 10 mg; or celecoxib 550 mg+rupatadine 12.5 mg, or celecoxib 550 mg+rupatadine 15 mg; or celecoxib 550 mg+rupatadine 17.5 mg; or celecoxib 550 mg+rupatadine 20 mg; or celecoxib 600 mg+rupatadine 2.5 mg; or celecoxib 600 mg+rupatadine 5 mg, or celecoxib 600 mg+rupatadine 7.5 mg; or celecoxib 600 mg+rupatadine 10 mg; or celecoxib 600 mg+rupatadine 12.5 mg, or celecoxib 600 mg+rupatadine 15 mg; or celecoxib 600 mg+rupatadine 17.5 mg; or celecoxib 600 mg+rupatadine 20 mg.

SSRI. Selective serotonin reuptake inhibitors (SSRI) are a class of drugs that function by increasing the extracellular level of the neurotransmitter serotonin by limiting its reabsorption (reuptake) into the presynaptic cell, increasing the level of serotonin in the synaptic cleft available to bind to the postsynaptic receptor. They have varying degrees of selectivity for the other monoamine transporters, with pure SSRIs having strong affinity for the serotonin transporter and only weak affinity for the norepinephrine and dopamine transporters. Examples of SSRI include, without limitation, citalopram, escitalopram, fluoxetine, paroxetine, and sertraline. In some embodiments an SSRI for use in the combination therapy disclosed herein is sertraline.

For the purposes of the present invention, an effective dose of sertraline or another SSRI in a combination with rupatadine is the dose that, when administered for a suitable period of time, usually at least about one week, and may be about two weeks, or more, up to extended periods of time of months or years, will reduce the progression of the disease. It will be understood by those of skill in the art that an initial dose may be administered for such periods of time, followed by maintenance doses, which, in some cases, will be at a reduced dosage.

The formulation and administration may generally follow conventional usage.

Bile acids. Bile acids are acids found predominantly in bile of mammals and other vertebrates. Diverse bile acids are synthesized in the liver. Bile acids are conjugated with taurine or glycine residues to give anions called bile salts. Primary bile acids are those synthesized by the liver. Secondary bile acids result from bacterial actions in the colon. In humans, taurocholic acid and glycocholic acid (derivatives of cholic acid) and taurochenodeoxycholic acid and glycochenodeoxycholic acid (derivatives of chenodeoxycholic acid) are the major bile salts. They are roughly equal in concentration.

The natural bile acid, chenodeoxycholic acid, was identified in 1999 as the most active physiological ligand for the farnesoid X receptor (FXR), which is involved in many physiological and pathological processes. The farnesoid-X-receptor (FXR) and the G protein bile acid receptor (GPBAR) 1 are two bile acid-activated receptors that exert regulatory effects on lipid, glucose, energy, and immune homeostasis. A series of alkylated bile acid analogues were designed and studied, with 6a-ethyl-chenodeoxycholic acid emerging as the most highly potent FXR agonist. FXR-dependent processes in liver and intestine were proposed as therapeutic targets in human diseases. Obeticholic acid is the first FXR agonist to be used in human drug studies.

Obeticholic acid (abbreviated to OCA, trade name Ocaliva), is a semi-synthetic bile acid analogue which has the chemical structure 6a-ethyl-chenodeoxycholic acid. It is used as a drug to treat primary biliary cholangitis, and is undergoing development for several other liver diseases and related disorders.

Deoxycholic acid, also known as cholanoic acid, and sold under the brand name Kybella among others, is a bile acid. Deoxycholic acid is one of the secondary bile acids, which are metabolic byproducts of intestinal bacteria. The two primary bile acids secreted by the liver are cholic acid and chenodeoxycholic acid. Bacteria metabolize chenodeoxycholic acid into the secondary bile acid lithocholic acid, and they metabolize cholic acid into deoxycholic acid. There are additional secondary bile acids, such as ursodeoxycholic acid. Deoxycholic acid is soluble in alcohol and acetic acid. When pure, it comes in a white to off-white crystalline powder form.

Ursodeoxycholic acid (UDCA), also known as ursodiol, is a secondary bile acid, produced in humans and most other species from metabolism by intestinal bacteria. It is synthesized in the liver in some species, and was first identified in bear bile, which is the derivation of its name Ursus. In purified form, it has been used to treat or prevent several diseases of the liver or bile ducts.

For the purposes of the present invention, an effective dose of OCA, ursodeoxycholic acid, deoxycholic acid or another bile acid in a combination with rupatadine is the dose that, when administered for a suitable period of time, usually at least about one week, and may be about two weeks, or more, up to extended periods of time of months or years, will reduce the progression of the disease. It will be understood by those of skill in the art that an initial dose may be administered for such periods of time, followed by maintenance doses, which, in some cases, will be at a reduced dosage.

The formulation and administration may generally follow conventional usage.

Metformin. Metformin is in the biguanide class of antidiabetic medications, which also includes the withdrawn agents phenformin and buformin. Metformin is sold under the brand name Glucophage among others, is the medication for the treatment of type 2 diabetes, particularly in people who are overweight. It is not associated with weight gain and is taken by mouth. Metformin is generally well tolerated. Common adverse effects include diarrhea, nausea, and abdominal pain. It has a low risk of causing low blood sugar. High blood lactic acid level is a concern if the medication is used in overly large doses or prescribed in persons with severe kidney problems. It is not recommended in those with significant liver disease. Metformin is a biguanide antihyperglycemic agent. It works by decreasing glucose production by the liver, by increasing the insulin sensitivity of body tissues, and by increasing GDF15 secretion, which reduces appetite and caloric intake.

The formulation and administration may generally follow conventional usage.

In some embodiments, the second agent is another selective H1 receptor fexofenadine, cetirizine, loratadine, astemizole, ketotifen, mizolastine acrivastine, ebastine, bilastine, bepotastine, terfenadine, or quifenadine.

The second agents, or combination of agents, can be incorporated into a variety of formulations for therapeutic administration. More particularly, the agents can be formulated into pharmaceutical compositions by combining them with appropriate pharmaceutically acceptable carriers or diluents either alone or in combination with an rupatadine, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. Oral formulations may be preferred.

Those of skill will readily appreciate that dose levels can vary as a function of the specific compound, the severity of the symptoms, and the susceptibility of the subject to side effects. Some of the specific compounds are more potent than others. Preferred dosages for a given compound are readily determinable by those of skill in the art by a variety of means. A preferred means is to measure the physiological potency of a given compound. The use of combination therapy may allow lower doses of each monotherapy than currently used in standard practice while achieving significant efficacy, including efficacy greater than that achieved by conventional dosing of either monotherapy, e.g. providing for a synergistic effect.

### Combinations and Formulations

A combination drug product of the invention, which can be provided as a single formulation or as two separate formulations of the active ingredients, a selective histamine H1 receptor antagonist, e.g. rupatadine, drug combination and a pharmaceutically acceptable excipient is provided, for example in combination of a COX-2 inhibitor, e.g. celecoxib; in combination with a fibrate, e.g. fenofibrate; in combination with a PPARα antagonist, e.g. fenofibrate or TPST-1120; in combination with an SSRI, e.g. sertraline; in combination with a bile acid, e.g. OCA; in combination with a second selective antagonist of histamine H1 receptor, e.g. cetirizine; etc. In preferred embodiments the combination provides for a synergistic improvement in disease markers or disease symptoms over the administration of either drug as a single agent.

In some embodiments, the formulation or combination of active agents consists essentially of a selective histamine H1 receptor antagonist, e.g. rupatadine, drug combination and a pharmaceutically acceptable excipient, for example in combination of a COX-2 inhibitor, e.g. celecoxib; in combination with a fibrate, e.g. fenofibrate; in combination with a PPARα antagonist, e.g. TPST-1120; in combination with an SSRI, e.g. sertraline; in combination with a second selective antagonist of histamine H1 receptor, e.g. cetirizine; etc, i.e. no additional active agents are included in the formulation, although excipients, packaging and the like will be present.

The combination can be defined based on the weight ratio of the two drugs, where the ratio may range from about 1:1, or 2:1, or 5:1, or 10:1, or 20:1 to 60:1, or from about 3:1 to 50:1, or from about 5:1 to 10:1, or from about 15:1 to 20:1.

For demonstrating the synergistic activity of the two drugs and establishing an appropriate fixed-dose ratio for clinical investigation, varying amounts of the two drugs are administered to appropriate animal models of inflammatory disease, either at a time of active disease (following disease onset) or at an early time point representative of preclinical disease, and the effect on disease activity or progression is measured. Alternatively, the effects of varying amounts of the two drugs are tested on a cellular response mediating inflammation that may be involved in the pathogenesis of disease.

It is within the level of skill of a clinician to determine the preferred route of administration and the corresponding dosage form and amount, as well as the dosing regimen, i.e., the frequency of dosing. In general terms it is most likely that the choice will be between once-a-day (s.i.d.) dosing and twice-a-day (b.i.d.) dosing. However, this generalization does not take into account such important variables as the specific type of articular cartilage or subchondral bone degeneration or destruction involved, the specific therapeutic agent involved and its pharmacokinetic profile, and the specific individual involved. For an approved product in the marketplace, much of this information is already provided by the results of clinical studies carried out to obtain such approval. In other cases, such information may be obtained in a straightforward manner in accordance with the teachings and guidelines contained in the instant specification taken in light of the knowledge and skill of the artisan. The results that are obtained can also be correlated with data from corresponding evaluations of an approved product in the same assays.

In one aspect, the present invention provides a unit dosage form of the formulation of the invention. The term “unit dosage form,” refers to physically discrete units suitable as unitary dosages for human subjects, each unit containing a predetermined quantity of drugs in an amount calculated sufficient to produce the desired effect in association with a pharmaceutically acceptable diluent, carrier, or vehicle. The specifications for the unit dosage forms of the present invention depend on the particular combination employed and the effect to be achieved, and the pharmacodynamics associated with the host.

In one aspect, the agents are formulated into pharmaceutical compositions by combination with appropriate, pharmaceutically acceptable carriers or diluents, and are formulated into preparations in solid, semi-solid, liquid, suspension, emulsion, or gaseous forms, such as tablets, capsules, powders, granules, ointments, solutions, suspensions, emulsions, suppositories, injections, inhalants, gels, microspheres, and aerosols. As such, administration can be achieved in various ways, usually by oral administration. In pharmaceutical dosage forms, the drugs may be administered in the form of their pharmaceutically acceptable salts, or they may also be used alone or in appropriate association, as well as in combination with other pharmaceutically active compounds. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch, or potato starch; with binders, such as crystalline cellulose, cellulose derivatives, acacia, corn starch or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Pharmaceutically acceptable excipients, such as vehicles, adjuvants, carriers or diluents, are commercially available. Moreover, pharmaceutically acceptable auxiliary substances, such as pH-adjusting and buffering agents, tonicity-adjusting agents, stabilizers, wetting agents and the like, are commercially available. Any compound useful in the methods and compositions of the invention can be provided as a pharmaceutically acceptable base-addition salt. “Pharmaceutically acceptable base-addition salt” refers to those salts that retain the biological effectiveness and properties of the free acids, which are not biologically or otherwise undesirable. These salts are prepared by adding an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline, and caffeine.

As used herein, compounds that are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

Although specific drugs are exemplified herein, any of a number of alternative drugs and methods apparent to those of skill in the art upon contemplation of this disclosure are equally applicable and suitable for use in practicing the invention. The methods of the invention, as well as tests to determine their efficacy in a particular patient or application, can be carried out in accordance with the teachings herein using procedures standard in the art. Thus, the practice of the present invention may employ conventional techniques of molecular biology (including recombinant techniques), microbiology, cell biology, biochemistry, and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass humans having pre-or early-stage inflammatory disease. Subjects may be human, but also include other mammals, particularly those mammals useful as laboratory models for human disease, e.g. mouse, rat, cats, dogs, horses, etc.

The expression “body fluid” as used herein in intended to include all of those accessible body fluids usable as clinical specimens which may contain a compound being tested for in sufficient concentration in said fluid to be within the limits of detection of the test device or assay being used. Body fluids will thus include whole blood, serum, plasma, urine, cerebrospinal fluid, synovial fluid, and interstitial and other extracellular fluids, particularly synovial fluid of affected joints. In some embodiments a body fluid used for determination of a marker of early-stage inflammation is a synovial fluid from a joint suspected of being involved in early arthritis. In other embodiments a body fluid used for marker determination is systemic, e.g. blood, urine, etc.

Care should be exercised in the collection and storage of the fluids to be tested. Steps should be taken to avoid proteolysis of the compounds to be tested for in said fluids, and freezing of the fluids is usually warranted unless the test involved can be carried out within a shortly after the fluids are collected. It is usually preferable to use synovial fluid rather than serum because of the likelihood that there will be greater concentrations of the compounds being tested for in the synovial fluid. On the other hand, increased levels of viscosity in synovial fluids pose problems in immunoassay systems that must be addressed by the artisan. It may be preferable to conduct longitudinal studies of a selection of cytokines and markers as well as their respective inhibitors and binding proteins in order to obtain the most accurate profile possible in determining whether an individual is in the early stages of articular cartilage degeneration and is therefore a candidate for intervention with the methods of the invention.

For the purposes of the present invention, “remodeling of subchondral bone” encompasses the variety of changes in subchondral and adjacent bone that occur in OA and in RA. For OA, “remodeling of subchondral bone” encompasses the presence of one or more of the following: development of subchondral sclerosis, development of subchondral cysts, development of ectopic bone formation (termed osteophytes), and other changes of the subchondral and adjacent bone that occurs in pre-clinical OA, early-stage OA, and established OA (Weinans et al, Bone. 2012 August; 51(2): 190-6. PMID: 22343134; Baker-LePain et al, Bone. 2012 August; 51(2): 197-203. PMID: 22401752). For RA, “remodeling of subchondral bone” encompasses the presence of one or more of the following: development of periarticular erosions, development of other bone erosions, development of justa-articular osteopenia, and other changes of the subchondral and adjacent bone that occur in preclinical RA, early-stage RA, and established RA (Deal C. Curr Rheumatol Rep. 2012 June; 14(3): 231-7. PMID: 22527950).

### Treatment

The methods of the invention can be used for prophylactic as well as therapeutic purposes. As used herein, the term “treating” refers both to the prevention of pre-clinical or early-stage inflammatory disease, and the treatment on ongoing disease. The invention provides a significant advance in the treatment of early-stage ongoing disease, by preventing the development of clinical symptoms of a disease or by preventing progression of the clinical symptoms of a disease. Such treatment is desirably performed prior to loss of function in the affected tissues, i.e. in early disease or pre-clinical disease states.

This invention specifically provides for the treatment of humans and other mammals that have pre-clinical or early-stage inflammatory disease but are asymptomatic. In such asymptomatic individuals with pre-clinical or early-stage inflammatory disease, this invention can prevent the development of symptomatic inflammatory disease or reduce the progression of symptomatic inflammatory disease.

This invention also specifically provides for the treatment of humans and other mammals that have early-stage (which in certain cases and diseases can be symptomatic) or established-inflammatory disease. In such symptomatic individuals with early-or established inflammatory disease, this invention can prevent progression of or reduce the severity of the inflammatory disease.

The expression “presently or prospectively” as used herein is intended to mean that in accordance with the methods discussed below of making that determination, it is possible to identify an individual as either being presently in need of such treatment, or very likely or expected to be in need of such treatment in the near-term future. Prospective need of treatment may be established by those determinations of positive factors that from the experience of the artisan lead directly to the early stages of an inflammatory disease.

The expression “the early stages of inflammatory disease” is intended to mean the very beginning of the initial pathologic changes. Said pathologic changes include changes in the composition, form, density, and/or inflammatory state of the involved tissue or organ from that present in healthy individuals.

Individuals with pre-clinical or early-stage inflammatory disease can be treated with a combination therapy of the invention to prevent the development of disease and to prevent the onset of symptomatic disease. The rupatadine and fibrate, etc. can be delivered in individual tablets or capsules, or in a combined tablet or capsule that includes both drugs. Importantly, this novel use of this combination does not require use of an antibiotic, an anti-viral or an anti-bacterial agent. No antibiotic, anti-viral, or anti-bacterial compound is needed for the anti-inflammatory activity and disease-modifying activity described herein.

Individuals at increased risk for development of an inflammatory disease, with early-stage inflammatory disease, or with established inflammatory disease, can be treated with a combination of the invention to prevent the development of disease as well as to prevent the progression of disease. The selective histamine H1 receptor antagonist, e.g. rupatadine, drug combination and a pharmaceutically acceptable excipient is provided, for example in combination of a COX-2 inhibitor, e.g. celecoxib; in combination with a fibrate, e.g. fenofibrate; in combination with another PPARα antagonist, e.g. TPST-1120; in combination with an SSRI, e.g. sertraline; in combination with a second selective antagonist of histamine H1 receptor, e.g. cetirizine; etc. and can be delivered in individual tablets or capsules or in a combined tablet or capsule that includes both drugs. Inflammatory diseases include degenerative diseases including osteoarthritis, Alzheimer's disease, macular degeneration and other degenerative diseases; autoimmune diseases including multiple sclerosis, rheumatoid arthritis, Crohn's disease, psoriasis and other autoimmune diseases; metabolic diseases including type II diabetes, coronary artery disease, metabolic syndrome and other metabolic diseases; chronic infections that result in inflammation including human immunodeficiency virus infection, hepatitis C virus infection, cytomegalovirus infection, and other viral, bacterial, fungal, parasite and other infection; and other inflammatory diseases such as fatty liver disease.

Of particular interest are inflammatory diseases associated with pain, including osteoarthritis (OA), rheumatoid arthritis (RA), juvenile rheumatoid arthritis (JRA), juvenile idiopathic arthritis (JIA), psoriatic arthritis (PSA), ankylosing spondylitis (AS), joint injury, dysmenorrhea and other conditions and diseases with an inflammatory component. In particular, the present invention relates to use of combination therapies to treat pain and/or slow disease progression.

In some embodiments, treatment of an individual with the methods disclosed herein decreases pain in the individual. In some embodiments, treatment of an individual with the methods disclosed herein decreases or stabilizes indicia of inflammation in the individual. In some embodiments, treatment of an individual with the methods disclosed herein decreases or stabilizes cartilage degeneration. Efficacy of therapy can be determined at a suitable timepoint after initiation of the treatment, for example after at least about 4 weeks, after at least about 8 weeks, after at least about 12 weeks, after at least about 3 months, after at least about 4 months, after at least about 5 months, after at least about 6 months, or more, e.g. up to about 9 months, up to about 1 year, up to about 2 years, etc.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, decrease in cartilage degeneration, decrease in pain, etc. (See, e.g., Physicians' Desk Reference (2010).)

Criteria for determining efficacy include, without limitation, an average of daily walking pain measured over a period of from 1 to 2 weeks; subject global assessment; WOMAC subscale and average scores; OMERACT-OARSI responder index; ICOAP score; EQ-5D-5L score; WPAI: OA score; RAND SF-36 score; physical activity tracked on the smartwatch; physician global assessment, etc.

In some embodiments the methods provide for at least a minimal clinically important improvement (MCII) in pain (see Tubach et al. (2005) Ann Rheum Dis 64: 29-33. doi: 10.1136/ard.2004.022905, herein specifically incorporated by reference. An MCII for knee OA pain is an improvement of at least 20 points (or 40%) on a visual analog scale (VAS) of 0-100. In some embodiments, treatment decreases a VAS score by more than 20 points, more than 25 points, more than 30 points, more than 35 points, mor than 40 points, more than 45 points, more than 50 points.

The decrease in pain following treatment may be at least a 10% decrease, at least a 20% decrease, at least a 30% decrease, at least a 40% decrease, at least a 50% decrease, at least a 60% decrease, at least a 70% decrease, at least a 80% decrease, at least a 90% decrease, or more. The improvement can be monitored by, for example and without limitation, daily walking pain averaged over 52 weeks; change from baseline in the WOMAC pain subscale, change from baseline in the WOMAC function subscale; change from baseline in the Patient's Global VAS; analysis of efficacy data using the OMERACT-OARSI Responder Index (Onel et al, Clin Drug Investig. 2008; 28 (1): 37-45. PMID: 18081359; etc.

The treatment can provide for a meaningful improvement in synovitis based on a reduction in the synovitis score (see, for example, Guermazi et al., Ann Rheum Dis. 2011 70(5): 805-11) as measured by Gd-MRI by greater than 3 points; greater than 4 points; greater than 5 points, greater than 6 points, or more.

### Treatment and Determination of an Individual With Pre-Clinical or Early-Stage OA

The present invention provides a method of treating or preventing degeneration or destruction of articular cartilage or remodeling of the subchondral bone in the joints of an individual in need of such treatment, comprising establishing the status of an individual as presently or prospectively being in said early stages and thus in need of such treatment; and administering to the individual a combined therapy of the invention in an amount therapeutically effective for treating or preventing said degeneration or destruction of articular cartilage or subchondral bone. In some embodiments the criteria for treatment further includes evidence of inflammation in the affected joint.

Assessment of OA may use the Kellgren Lawrence (KL) grading system (Kellgren and Lawrence, Ann. Rheum. Dis., 16: 494-502, 1957, herein specifically incorporated by reference). The KL grading system relies on an anterior-posterior (AP) radiograph and is as follows: grade 0=no features of OA; grade 1=presence of OA is doubtful, presence of minute osteophyte(s), unchanged joint space; grade 2=minimal OA, definite osteophyte(s), unchanged joint space; grade 3=moderate OA, moderate diminution of joint space; grade 4=severe OA, joint space greatly reduced with sclerosis of subchondral bone. For the purposes of the present invention, the KL score is less than 3, desirably less than 2, and in some embodiments is less than one.

Use of the combination therapies described herein is aimed at intervention during the pre-clinical or early stages of OA, during which there is evidence of only mild cartilage abnormalities or lesions as defined by the presence of at least one imaging marker indicative of pre-clinical or early-stage OA, as determined by imaging or direct visualization modalities, molecular marker analysis, or clinical history of a condition or event predisposing to the development of OA. The combination therapy of the invention modifies OA disease progression as measured by either stabilization of KL score and/or joint-space narrowing, or prevention of further cartilage breakdown (as assessed by imaging using MRI or another imaging modality), or reduction in levels of molecular markers of cartilage breakdown.

Individuals with pre-clinical or “pre-OA” are those at increased risk of developing OA, as evidenced by biochemical, imaging, or clinical markers. Conditions or events that predispose to the development of OA include, without limitation, a history of injury to a joint; clinically or radiographically diagnosed meniscal injury with or without surgical intervention; a ligamentous sprain with clinically or radiographically diagnosed anterior or posterior cruciate or medial or lateral collateral ligament injury (Chu et al, Arthritis Res Ther. 2012 14(3): 212. PMID: 22682469); clinically measured limb-length discrepancy; obesity with a current, or prolonged historical period of, BMI >27; or biomechanical features of abnormal gait or joint movement. In general, a determination of pre-clinical OA is associated with one or more, two or more, three or more parameters of joint pathology including, without limitation and relative to a healthy control sample, cartilage proteoglycan loss; cartilage damage; or elevated levels of degradative enzymes, the presence of products of cartilage or extracellular matrix degradation or bone remodeling. Humans at risk for OA, who have pre-OA, and who have early-stage OA are often asymptomatic, but a subset of patients experience joint pain due to cartilage injury (e.g. meniscal injury), ligamentous injury (e.g. tearing of the anterior cruciate ligament), or another joint abnormality.

Markers indicative of pre-clinical OA. Compared to the joints of healthy control individuals, a joint in an individual with pre-clinical OA will typically have one, two, three, four or more markers indicative of pre-clinical disease. MRI-detected imaging markers indicative of the presence of pre-clinical OA include cartilage edema, cartilage proteoglycan loss, cartilage matrix loss, bone marrow edema, articular cartilage fissures, articular cartilage degeneration, a meniscal tear, an anterior cruciate ligament tear, a posterior cruciate ligament tear, and other abnormalities of the cartilage or ligaments in the joint. Ultrasound will show evidence of cartilage edema or damage. Arthroscopy can allow direct detection or visualization of cartilage edema, cartilage softening, cartilage thinning, cartilage fissures, cartilage erosion, or other cartilage abnormalities. Cartilage damage is frequently defined by the Outerbridge classification criteria or similar directly observed changes within the joint. For example, one such scoring system defines the presence of damage is as follows: grade 0=normal cartilage; grade I: softening and swelling of cartilage; grade II: a partial-thickness defect in the cartilage with fissures on the surface that do not reach subchondral bone or exceed 1.5 cm in diameter; grade III: fissures in the cartilage that extend to the level of subchondral bone in an area with a diameter of more than 1.5 cm. Humans at risk for OA or with “pre-clinical OA” may be asymptomatic but may have signs of cartilage damage, meniscal damage, ligament damage, or other abnormalities of the joint.

Markers indicative of early-stage OA. As compared to joints in a healthy individuals, a joint in an individual with early-stage OA will typically have one, two, three, four or more markers indicative of early disease. Plain X-rays of the involved joint would demonstrate features consistent with a KL score of 1-3, including small osteophytes and no or minimal joint space narrowing. MRI-detected imaging markers indicative of early-stage OA include bone marrow lesions, bone marrow edema, cartilage proteoglycan loss, cartilage thinning, cartilage fissures or cartilage breakdown. Ultrasound will show evidence of cartilage, bone or synovial edema or damage. Arthroscopy can provide for direct detection or visualization of cartilage edema, cartilage softening, cartilage thinning, cartilage fissures, cartilage erosion, or other cartilage abnormalities. Cartilage damage is frequently defined by the Outerbridge classification criteria or similar direct observational changes within the joint. Humans with early OA may be asymptomatic but may experience joint pain. They may also exhibit findings associated with cartilage damage as represented by Outerbridge grade 0, grade I and grade II scores or similar direct observational changes within the joint, as well as with other cartilage, meniscal and ligament damage.

Established and Advanced OA. In contrast to pre-clinical OA and early-stage OA, advanced OA can be defined radiographically as KL grade>=3 or as MRI evidence of extensive, complete, or near-complete loss of articular cartilage. Other evidence of joint failure can be determined by direct or arthroscopic visualization of extensive, complete, or near-complete loss of joint space or cartilage, by biomechanical assessment of inability to maintain functional joint integrity, or by clinical assessment of joint failure, as evidenced by inability to perform full range of motion or to maintain normal joint function. Patients with advanced OA frequently experience joint pain. On physical examination, patients with advanced OA can have bony enlargement, small effusions, crepitus, and malalignment of the synovial joints. Examples of semiquantitative MRI scoring systems that can be used to classify the severity of OA include: WORMS (Whole-Organ Magnetic Resonance Imaging Score; Peterfy CG, et al. Osteoarthritis Cartilage 2004; 12:177-190); KOSS (Knee Osteoarthritis Scoring System; Kornaat PR, et al. Skeletal Radiol 2005; 34:95-102); BLOKS (Boston Leeds Osteoarthritis Knee Score; Hunter DJ, et al. Ann Rheum Dis 2008; 67:206-211); MOAKS (MRI Osteoarthritis Knee Score; Hunter DJ, et al. Osteoarthritis Cartilage. 2011; 19(8): 990-1002); HOAMS (Hip Osteoarthritis MRI Score; Roemer FW, et al. Osteoarthritis Cartilage. 2011; 19(8): 946-62); OHOA (Oslo Hand Osteoarthritis MRI Score). Advanced OA can result in significant joint pain and loss of mobility owing to joint dysfunction. In preferred embodiments the individual treated by the methods of the invention has preclinical or early-stage arthritis rather than advanced OA or RA.

Assessing inflammation in preclinical OA, early-stage OA, and advanced OA.

A variety of markers can be used to assess inflammation in preclinical OA, early-stage OA, and advanced OA, including imaging markers, molecular markers, and clinical markers. Examples of clinical markers include the presence of a joint effusion on physical examination. Another example of a clinical marker is the presence of morning stiffness in the joint. Examples of imaging markers include the use of MRI or ultrasound-detected signs of inflammation in the joint. MRI can be performed either with or without gadolinium contrast, and MRI-evidenced inflammation is defined as the presence of one or more of the following findings: synovitis (synovial lining thickening, proliferation, and/or enhancement (increased signal), including a positive Doppler-flow signal in the synovial lining), joint effusion, bone marrow edema, etc (Krasnokutsky et al, Arthritis Rheum. 2011 63(10): 2983-91. doi: 10.1002/art.30471 PMID: 21647860; Roemer et al, Osteoarthritis Cartilage. 2010 October; 18(10): 1269-74. PMID: 20691796; Guermazi et al, Ann Rheum Dis. 2011 70(5): 805-11, PMID: 21187293). Ultrasound-evidenced inflammation is defined as the presence of one or more of the following findings: synovial lining thickening and/or enhancement, a joint effusion, bone marrow enhancement, etc. (Guermazi et al, Curr Opin Rheumatol. 2011 23 (5): 484-91. PMID: 21760511; Hayashi et al, Osteoarthritis Cartilage. 2012 March; 20(3): 207-14. PMID: 22266236; Haugen et al, Arthritis Res Ther. 2011; 13(6): 248. PMID: 22189142). Molecular markers that can be used to assess inflammation include erythrocyte sedimentation rate (ESR), CRP, cytokines, chemokines, and other inflammatory mediators. ESR and CRP are measured in blood, and the other molecular markers of inflammation can be measured in blood or synovial fluid.

### Assessing Inflammation in Preclinical Disease, Early-Stage Disease, and Established Disease

The following provides examples of approaches to determining whether inflammation is present in an individual, including individuals at risk for a variety of different inflammatory diseases, such as autoimmune diseases (e.g., RA, MS, Crohn's disease, psoriasis, etc), degenerative diseases involving low-grade inflammation (e.g., OA, Alzheimer's disease, macular degeneration, etc), other inflammatory diseases (e.g., NASH, type II diabetes, metabolic syndrome, atherosclerosis, cardiac disease, etc), as well as inflammatory diseases associated with chronic inflammation (e.g., HIV infection, HCV infection, CMV infection, TB infection, etc). Although the following describes the approach to identifying inflammation particularly in humans at risk of developing arthritis or with early-stage arthritis, variations of this approach are generally applicable to humans with a wide-range of inflammatory diseases.

A variety of markers can be used to assess inflammation in inflammatory diseases, including imaging markers, molecular markers, and clinical markers. Examples of clinical markers include warmth, erythema (redness), synovitis, joint effusions. Other examples of clinical markers are morning stiffness in the joint lasting more than 1 hour, and pain and swelling. Examples of imaging markers include MRI-or ultrasound-detected inflammation in the joint. MRI, performed with or without gadolinium contrast, detects inflammation on the basis of the presence of one or more of the following findings: synovitis (synovial lining thickening, proliferation and/or enhancement (increased signal on Gd-MRI)); increased Doppler-flow signal in the synovial lining); a joint effusion; extensive bone marrow edema; and other findings suggestive of inflammation. When ultrasound is the imaging method used, inflammation is defined by the presence of one or more of the following findings: synovial lining thickening and/or enhancement, a joint effusion, bone marrow enhancement, and other findings suggestive of inflammation. Molecular markers that can be used in assessing inflammation include ESR, CRP, cytokines, chemokines, and other inflammatory mediators. ESR and CRP are measured in blood, and the other molecular markers of inflammation can be measured in blood or synovial fluid. Use of molecular markers in blood for identifying individuals with preclinical RA or early-stage RA is described in Sokolove et al. (PLOS One. 2012; 7(5): e35296, PMID: 22662108) and Deane et al. (Arthritis Rheum. 2010 62(11): 3161-72. doi: 10.1002/art.27638. PMID: 20597112).

The presence of early-stage arthritis, including early-stage OA and RA, may also be determined or confirmed by a difference in level of a molecular marker in body fluids, including without limitation synovial fluid, or joint tissue relative to that in a control body fluid or joint tissue that is free of arthritis, or in blood, or in urine. Examples of such changes in levels of molecular marker are the following: increase in level of interleukin-1 beta (IL-1β); increase in level of TNF; increase in ratio of IL-1β to IL-1 receptor antagonist protein (IRAP); increase in expression of p55 TNF receptors (p55 TNF-R); increase in level of interleukin-6 (IL-6); increase in level of leukemia inhibitory factor (LIF); altered levels of insulin-like growth factor-1 (IGF-1), increase in levels of transforming growth factor beta (TGFB), platelet-derived growth factor (PDGF), or basic fibroblast growth factor (b-FGF); increase in level of keratan sulfate; increase in level of stromelysin; increase in ratio of stromelysin to tissue inhibitor of metalloproteases (TIMP); increase in in level of osteocalcin; increased alkaline phosphatase; increased CAMP responsive to hormone challenge; increased urokinase plasminogen activator (uPA); increase in level of cartilage oligomeric matrix protein; other cartilage breakdown products including CTX-II; increase in level of collagenase; increase in level of other cytokines; increase in in level of CRP; or increase in in level of autoantibodies against synovial joint proteins or other biomolecules. The term “metalloprotease” as used herein is intended to refer to MMPs, especially those whose levels are typically elevated concentrations where there is articular cartilage degeneration, i.e., stromelysins, collagenases, and gelatinases. Aggrecanase is also included within this term. The three collagenases present in articular cartilage during the early stages of degeneration are collagenase-1 (MMP-1), collagenase-2 (MMP-8), and collagenase-3 (MMP-13). Of the three stromelysins, stromelysin-1 (MMP-3), stromelysin-2 (MMP-10), and stromelysin-3 (MMP-11), only stromelysin-1 appears in articular cartilage during the early stages of its degeneration. The metalloproteases are secreted by chondrocytes as proenzymes, which must be activated before they can degrade extracellular matrix macromolecules. Activation of these proenzymes involves an enzymatic cascade in which serine proteases, including the plasminogen activator-plasmin system, play a key role.

IL-1, which exists as IL-1α and IL-1β, is a catabolic cytokine that mediates articular cartilage injury and loss in mammalian joints. It suppresses the synthesis of type II collagen found in articular cartilage, while promoting the synthesis of type I collagen characteristic of fibroblasts; induces the production of enzymes involved in matrix degradation; and suppresses the ability of chondrocytes to synthesize new proteoglycans. IL-1 and its modulator IRAP are produced in an autocrine and paracrine fashion by synovial macrophages, and IRAP production may increase in the presence of granulocyte macrophage colony-stimulating factor (GM-CSF). However, IL-1 is much more potent than IRAP, with approximately 130-fold more IRAP being required to abolish the pathogenic effects of IL-1, as measured in chondrocytes and cartilage explants. Imbalances between IL-1 and IRAP exacerbates the degeneration of articular cartilage. Consequently, it is also appropriate to identify abnormalities in the levels of IL-1 and IRAP, as well as in the ratio of IL-1 to IRAP, to identify an individual in the early stages of cartilage injury and loss before focal cartilage loss can be identified radiographically. Thus, determining the levels of IL-1 and IRAP, as well as the ratio of IL-1 to IRAP, could enable identification of individuals that are candidates for early pharmacological intervention before significant cartilage degeneration occurs. Furthermore, the frequency of IL-1α- and IL-1β-secreting macrophages is significantly greater in the synovial fluid and synovial tissue of joints undergoing the early stages of articular cartilage degeneration can be detected and is significantly greater than in synovial fluid and synovial tissue from normal joints, i.e., joints in which there is no articular cartilage degeneration.

In mammals subjected to sectioning of the cruciate ligament of a knee joint, the concentration of TNF is significantly higher in the synovial fluid of the sectioned knee joint than in that of the contralateral, unsectioned knee joint. The expression of p55 TNF receptors (TNF-R) on chondrocytes in articular cartilage is also higher in the sectioned knee joint. Therefore, because an increase in TNF levels, and possibly TNF signaling, is associated with early cartilage injury and loss, it is appropriate to measure levels of TNF and TNF-R in the joints of individuals at risk for cartilage degeneration and loss. These results contribute to diagnostic classification of individuals that are candidates for early pharmacological intervention before significant cartilage degeneration occurs.

IL-6 is an inflammatory cytokine whose are abnormally high in the joints and synovial fluid of damaged limbs. IL-6 increases the expression of TNF-R on chondrocytes and the production of proteoglycan by chondrocytes; it also induces the release of glycosaminoglycans from the cartilage matrix. Comparing IL-6 levels in synovial fluid and chondrocytes of joints in the early stages of articular cartilage injury and loss to that in synovial fluid and chondrocytes of control joints can identify individuals that are appropriate candidates for pharmacological treatment, before any focal cartilage loss is detectable by radiographic examination.

LIF is produced by monocytes, granulocytes, T cells, fibroblasts, and other cell types associated with inflammatory conditions. Synoviocytes and chondrocytes synthesize and secrete LIF in the presence of IL-1β and TNFα. Thus, identifying increases in levels of LIF can allow selection of candidates for pharmacologic treatment of the early stages of articular cartilage injury and loss.

IGF exists as types I and II, and IGF-I mediates cartilage synthesis. Furthermore, it reduces degradation and promotes synthesis of proteoglycans even in the presence of IL-1β and TNFα. Serum levels of IGF-1 are maintained by high-affinity binding proteins (IGF-BPs), and IGF-1 regulates both bone and cartilage turnover. Detecting abnormally high levels of IGF-1 permits identification of candidates for early pharmacologic treatment of articular cartilage degeneration.

TGFB is produced by chondrocytes and acts as a powerful mitogen contributing to the turnover of both cartilage and bone. Further, it stimulates the synthesis of extracellular matrix and has anti-inflammatory activity. It also inhibits the degradation of the extracellular matrix by stimulating the production of protease inhibitor, and blocking the release of collagenases and metalloproteases. Further still, it promotes cartilage repair by stimulating production of collagen, fibronectin, inhibitors of plasminogen activators, and tissue inhibitors of metalloproteases (TIMP) by various cells in the mammal joint. Synovial fluid levels of TGFB are abnormally low in the joints of mammals in the early stages of articular cartilage injury and loss. Consequently, levels of TGFB compared to control permit diagnostic evaluation of candidates for early pharmacologic treatment of articular cartilage degeneration.

With progressive degeneration, i.e., catabolismof the articular cartilage in the joint, a number of metabolites are produced that are useful as markers of the cartilage degeneration, both to the occurrence and to the progression of cartilage degeneration. For example, IL-1a and IL-1β or TNFα active inflammatory and degradative pathways that mediate cartilage degradation and release of glycosaminoglycans (GAGS), which can be measured in the synovial fluid of an individual. Furthermore, GAG levels change after treatment so that it is possible to monitor the efficacy of pharmacologic intervention, by using GAG levels in synovial fluid as a marker of articular cartilage turnover. Because the degradation of articular cartilage involves collagen as well as the other cartilage components, several collagen breakdown products serve as markers of cartilage degradation in mammals. Type-Il-specific collagen breakdown products, e.g., 20-30 amino acid neoepitopes, can be identified in body fluids such as synovial fluid, plasma, serum, and urine. The presence of collagen neoepitopes in these body fluids may be used as indicators of OA onset and progression.

The presence or an increase in the levels of 5D4, a neo-epitope of the GAG keratan sulfate, in synovial fluid is a marker of early articular cartilage injury and loss. Conversely, presence of or increased levels of various neo-epitopes of chondroitin sulfate, another GAG, is associated with anabolic events in the articular cartilage of mammals in the early stages of cartilage injury and loss. Levels of these epitopes in synovial fluid, particularly 3B3, 7D4 and 846, can be determined by specific monoclonal antibodies. The 3B3 epitope is expressed on chondroitin sulfate chains of cartilage during repair and remodeling of the extracellular matrix, and consequently its levels in synovial fluid correlate inversely with those of 5D4. The determination of 3B3 levels in the synovial fluid of test mammals and comparison of these levels with control values permits the creation of a diagnostic profile of a mammal that is an appropriate candidate for early pharmacologic treatment.

Additional markers of cartilage anabolism are the propeptides of type II procollagen (PIIP). Type II collagen is the major collagen of articular cartilage and is produced by chondrocytes as the procollagen PIIP. During the process of collagen fibril formation, aminopropeptide and carboxypropeptide, the noncollagenous portions of PIIP, are cleaved and released into body fluids, where they can be measured as a reflection of anabolic activity in articular cartilage. Levels of the carboxypropeptide of PIIP (carboxy-PIIP) in synovial fluid are higher during cartilage anabolism and correlate with radiographic evidence of pathologic changes in the cartilage. Accordingly, detection of increased levels of carboxy-PIIP in synovial fluid identifies individual for early pharmacologic treatment.

Perturbation of the stromelysin: TIMP ratio in articular cartilage and joint fluids of mammals is another marker of early-stage articular cartilage degeneration. Abnormal joint loading after joint injury causes the production of excess stromelysin, an enzyme produced by chondrocytes and synoviocytes in an IL-1-mediated process. The concentrations of stromelysin are higher in fibrillated (injured) cartilage than they are in cartilage more distal to the injury. Levels of stromelysin may be excessively high for only a short period of time, but where the damage to the joint transcends the tidemark zone of the articular cartilage and reaches into the subchondral bone, there is a substantial likelihood of subsequent articular cartilage degeneration, usually preceded by a stiffening of the subchondral bone. In such situations, there is an increased number of cells involved in the synthesis of stromelysin, IL-1α, IL-1β, and the oncogene proteins c-MYC, c-FOS, and c-JUN. In the synovium cells that secrete these factors are the superficial synovial lining cells, while in the cartilage such cells are the chondrocytes in the superficial and middle layers and the condrocytes in the fibrillated areas of the tibial plateau. Further, stromelysin and IL-1 diffuse into the cartilage matrix of the tibial plateau. Stromelysin, which degrades components of connective tissue, including proteoglycans and type IX collagen, is actively synthesized in the synovium of mammals in the early stages of articular cartilage degeneration, and is the primary proteolytic enzyme involved in the cartilage destruction. Increased levels of stromelysin mRNA are detectable in the synovia of such mammals, as are increased levels of collagenase mRNA. Increased levels of both isoforms of IL-1, but especially IL-1β, stimulate the synthesis of stromelysin and collagenase by synovial fibroblasts. IL-1 does not stimulate the production of tissue inhibitor of metalloprotease (TIMP), such that the levels of this metalloprotease inhibitor in the synovium remain unchanged while the levels of metalloproteases are dramatically increased.

### Assessment of Biomarkers for Determination of a Condition Eligible for Treatment

Various techniques and reagents can be used in the analysis of biomarkers in the present invention. In one embodiment of the invention, blood or synovial fluid samples, or samples derived from blood, e.g. plasma, serum, etc., are assayed for the presence of specific biomarkers. Other sources of samples are body fluids such as synovial fluid, lymph, cerebrospinal fluid, bronchial aspirates, saliva, milk, urine, and the like. Also included are derivatives and fractions of such cells and fluids. Diagnostic samples are collected any time that an individual is suspected of having an inflammatory disease or of being at risk of developing an inflammatory disease. Such assays come in many different formats, including autoantigen arrays; enzyme-linked immunosorbent assays (ELISA) and radioimmunoassays (RIA); assays in which binding of labeled peptides in suspension or solution are measured by flow cytometry or mass spectrometry.

Many such methods are known to one of skill in the art, including ELISA, fluorescence immunoassays, protein arrays, eTag system, bead-based systems, tag or other array-based systems, surface plasmon resonance (SPR)-based detection systems, etc. Examples of such methods are set forth in the art, including, inter alia, chip-based capillary electrophoresis: Colyer et al. (1997) J Chromatogr A. 781(1-2): 271-6; mass spectroscopy: Petricoin et al. (2002) Lancet 359: 572-77; eTag systems: Chan-Hui et al. (2004) Clinical Immunology 111: 162-174; microparticle-enhanced nephelometric immunoassay: Montagne et al. (1992) Eur J Clin Chem Clin Biochem. 30(4): 217-22; the Luminex XMAP bead-array system (www.luminexcorp.com); and the like, each of which are herein incorporated by reference.

For multiplex analysis, arrays containing one or more detection antibodies that recognize biomarkers of interest can be generated. Various immunoassays designed to quantitate the biomarkers may be used in screening. Measuring the concentration of the target protein or other biomarker in a sample or fraction thereof may be accomplished by a variety of specific assays. For example, a conventional sandwich-type assay may be used in an array, ELISA, RIA, bead array, etc. format.

Analysis of a biological sample may be done by using any convenient protocol, for example as described below. The readout may be a mean, average, median or the variance or other statistically or mathematically derived value associated with the measurement. The readout information may be further refined by direct comparison with the corresponding reference or control readout.

Following quantitation of the biomarker in the sample being assayed, the value obtained is compared with a reference or control value to make a diagnosis regarding the phenotype of the patient from whom the sample was obtained. Typically a comparison is made with the analogous value obtained from a sample or set of samples from an unaffected individual. Additionally, a reference or control value may be a value that is obtained from a sample of a patient known to have an autoimmune or degenerative disease of interest, such as RA or OA, and therefore may be a positive reference or control profile.

For prognostic purposes, an algorithm can be used that combines the results of determinations of multiple antibody specificities and/or cytokine levels, and/or levels of cartilage degeneration markers, and/or other markers, and that will discriminate robustly between individuals with autoimmune disease, e.g. RA, or degenerative disease, e.g. OA, and controls.

Included as a biomarker of inflammation is C reactive protein (CRP), including high-sensitivity CRP (hs-CRP). It is known that individuals with high levels of hs-CRP, even at the high end of the normal range, have 1.5 to 4 times increased risk of developing an inflammatory disease, including but not limited to atherosclerotic disease, atherosclerotic cardiovascular disease, RA, psoriatic arthritis, systemic lupus erythematosus, osteoarthritis, type II diabetes, metabolic syndrome, NAFLD, NASH and other inflammatory metabolic diseases.

The range of levels of plasma fibrinogen that is deemed normal varies from laboratory to laboratory but is typically 1.5-4.0 g/L. Levels of plasma fibrinogen above 2.8 g/L are associated with increased risk of developing an inflammatory disease, and levels >4 g/L are associated with an even higher risk.

Normal levels of serum amyloid A (SAA) range widely. However, elevations in SAA levels have been associated with increased risk with moderate elevation >3.9 but <8 mg/L.) conferring increase risk over the lowest tercile and values greater than 8.2 mg/L (highest tercile) imparting highest risk.

There is a wide range in ESR values that are considered normal, but ESR values suggestive of inflammation include >15 mm/hr in men under 50 years old, >20 in men over 50 and women under 50, and >30 mm/hr in women over 50.

MRI, with or without gadolinium or other contrast enhancement, can be used to detect the presence of inflammation and thereby identify individuals with an inflammatory disease or at increased risk of developing an inflammatory disease. For example, MRI-detected inflammation is defined by the presence of one or more of the following findings: synovitis (synovial lining thickening, proliferation and/or enhancement), a joint effusion, bone marrow edema, and other MRI imaging findings suggestive of inflammation (Krasnokutsky et al, Arthritis Rheum. 2011 63(10): 2983-91. doi: 10.1002/art.30471 PMID: 21647860; Roemer et al, Osteoarthritis Cartilage. 2010 October; 18(10): 1269-74. PMID: 20691796; Guermazi et al, Ann Rheum Dis. 2011 70(5): 805-11, PMID: 21187293). Guermazi et al. (Guermazi et al, Ann Rheum Dis. 2011 70(5): 805-11,

PMID: 21187293) defines a semiquantiative scoring system for grading the level of inflammation in joints, allowing one to determine (1) whether an individual has inflammation or not, and (2) the degree of inflammation in an individual. Individuals with evidence of joint inflammation according to the Guermazi scoring system can be classified as having increased risk for the development of OA, pre-clinical OA, early-stage OA, or established OA. The degree of inflammation as evaluated by the Guermazi scoring system predicts development and/or progression of the inflammatory disease OA. MRI, with or without gadolinium, can be applied to many other conditions to determine whether or not inflammation is present, and whether an individual with inflammation has pre-clinical inflammatory disease, early-stage inflammatory disease, or established inflammatory disease.

Ultrasound-detected inflammation is defined by the presence of one or more of the following findings: synovial lining thickening and/or enhancement, a joint effusion, bone marrow enhancement, a Doppler-flow signal in the synovial lining, and other findings suggestive of inflammation (Guermazi et al, Curr Opin Rheumatol. 2011 23(5): 484-91. PMID: 21760511; Hayashi et al, Osteoarthritis Cartilage. 2012 March; 20(3): 207-14. PMID: 22266236; Haugen et al, Arthritis Res Ther. 2011; 13(6): 248. PMID: 22189142).

In certain in vitro assays, ex vivo assays, and in vivo models, the combination of an rupatadine and a second agent, exhibits unexpected and surprising synergy in reducing the production of inflammatory mediators in in vitro and ex vivo assays, and in reducing disease activity and inflammation in in vivo models, and in reducing pain. In other in vitro assays, ex vivo assays, and in vivo models, the combination exhibits an unexpected and surprising additive effect in reducing the production of inflammatory mediators in in vitro and ex vivo assays, and reducing disease activity and inflammation in the in vivo model. In general, the individual agents administered alone did not provide as robust anti-inflammatory, pain-relieving, or disease-modifying activity as did the combinations.

### EXAMPLES

The following are examples of the methods and compositions of the invention. It is understood that various other embodiments may be practiced, given the general description provided above.

### Example 1

### Investigation of the Therapeutic Effects of Rupatadine, Fenofibrate, and Rupatadine Combinations in OA

We tested the relationship between beneficial activation of PPARA and inhibition of pathogenic PAF signaling by treating mice with fenofibrate, a PPARα agonist, and/or rupatadine, a PAF receptor and histamine H1 receptor. Following DMM, WT mice treated with either fenofibrate or rupatadine experienced significantly less severe OA-related cartilage degeneration as compared to vehicle-treated mice. Administration of both fenofibrate and rupatadine resulted in a further decrease in severity of cartilage degeneration as compared to vehicle or either drug alone.

Fenofibrate and Rupatadine treatment have synergistic effect in expression of PAF-related genes. We further examined the relationship between PPARA and PAF signaling by analyzing gene expression of related genes in human OA synovial tissue explants following treatment with fenofibrate and/or rupatadine. Human OA synovial tissues were stimulated with OA synovial fluids for 24 hours, followed by treatments with fenofibrate, rupatadine or both for another 24 hours. Following treatment with fenofibrate, LPCAT2 and PTAFR gene expression were reduced compared to SF-stimulated samples. Synergistic decreases in expression of LPCAT2 and PTAFR were seen following treatment with both fenofibrate and rupatadine, as compared to treatment with either drug alone. These findings suggest that PPARA expression has an inhibitory effect on PAF synthesis and uptake.

We demonstrated that levels of inflammatory lipids, including oxLDLs, acLDLs, and PAF, promote the development of OA. Treatment with fenofibrate, an FDA-approved PPARα agonist, and rupatadine, a dual antagonist of histamine receptor and PAF receptor may prevent OA through activating LDL metabolism to reduce inflammatory lipid generation such as PAF and by inhibiting NF-κB to reduce its activation of pro-inflammatory responses. Success of this demonstrates a critical role for the uptake, metabolism and modification of inflammatory lipids in OA, and identified FDA-approved and experimental therapeutics that could be rapidly translated into clinical trials for the prevention and treatment of OA.

TABLE 1
Lipid transport proteins are detected in human OA synovial fluids. Mass
spectrometric analysis of synovial fluids from 5 individuals with OA
revealed multiple lipid transport proteins including ApoE and LRP1.

| | | | % | # of |
|---|---|---|---|---|
| Accession # | Protein Name | Score | coverage | peptides |
| P02647 | Apolipoprotein A-I | 405.32 | 67 | 26 |
| P06727 | Apolipoprotein A-IV | 370.16 | 60 | 27 |
| P02649 | Apolipoprotein E | 49.32 | 21 | 4 |
| P02652 | Apolipoprotein A-II | 36.26 | 22 | 3 |
| Q07954 | Low-density Lipoprotein-related protein 1 | 14.54 | 1 | 2 |

Pharmacologic targeting of inflammatory lipid pathways in mouse OA to identify therapeutics to prevent and treat OA. Fibrates represent a class of lipid-targeting drugs, and have demonstrated efficacy in treating multiple types of cardiovascular diseases. Fenofibrate is an agonist of PPARα, a nuclear receptor that regulates lipid metabolism and is activated by the binding of fatty acids, resulting in reductions of serum triglyceride and LDL levels. We found that fenofibrate prevents the development of OA following DMM. qPCR analysis of synovial tissue from these mice showed that fenofibrate treatment resulted in decreased expression of multiple OA inflammatory mediators, consistent with the known function of PPARα to inhibit NF-KB-mediated expression of pro-inflammatory cellular responses. The failure of atorvastatin to prevent OA, and the efficacy of fenofibrate highlights the importance of gaining a full understanding of the molecular and cellular mechanisms by which inflammatory lipids promote the pathogenesis of OA, and then leveraging this mechanistic understanding to develop disease-modifying therapies for OA. Based on our findings to date, we believe that drugs that modulate lipid metabolism to reduce inflammatory lipid generation or their stimulation of synovial cells will prevent and treat OA. We will focus our study on FDA-approved drugs, and will test their abilities to prevent OA following DMM in mice. Gait and incapacitance analyses will monitor recovery of normal gait, behavior and pain (as surrogates for rehabilitation following joint injury in humans).

We measured the development of OA and functional outcomes following DMM in wild type B6 mice (n=10 per treatment group) treated with pharmacologic agents that: (i) activate PPAR-family function (including PPARα and/or PPARγ), thereby promoting LDL metabolism; (ii) inhibit CD36 and TLR2, thereby inhibiting uptake of inflammatory lipids; (iii) lower lipid levels through other mechanisms. Functional outcomes include (i) gait analysis using the CatWalk system, performed twice at baseline then monthly following DMM, and (ii) joint pain assessment by incapacitance testing to measure the static weight bearing of each hind paw simultaneously, performed 2x/week starting 1 week prior to DMM using a Incapacitance Meter. Radiographic outcomes will include X-ray and microCT performed at baseline, and then monthly following DMM. Histologic outcomes: 20 wks after DMM, mice are sacrificed, and OA assessed histologically by a blinded examiner scoring cartilage degeneration through Safranin-O stained sections, and scoring synovitis and osteophytes through H&E stained sections. Mechanistic analyses will be performed, including (i) immunostaining, (ii) qPCR transcriptional analysis, and (iii) biomarker analyses to characterize the mechanisms by which the efficacious candidate therapeutics prevent OA.

Treatment with pharmacologic agents. Beginning 24 hrs after DMM, mice (n=10 per experimental arm) are treated by injection or oral gavage for 12 wks with either vehicle, or one of the agents as detailed in Table 1. Small molecules will be administered by oral gavage (e.g. PO) or by IP injection based on previously described mouse dosing regimens at two dose levels.

DMM Mouse model of joint injury and PTOA. A 3-mm incision is made in one stifle joint, spanning the distal patella and the proximal tibial plateau in 16-to 20-wk-old WT male mice. The fat pad will be dissected to expose the medial meniscotibial ligament (MMTL), which will be severed. Contralateral stifle joints will be subjected to sham surgery (i.e., the joint capsule is incised but the MMTL not transected). 20-wks post-surgery, mice will be sacrificed, sham and DMM-operated joints harvested for histologic and mechanistic analysis.

### Mouse OA Assessment and Functional Outcomes

Functional gait analysis using the CatWalk System. Mice are monitored for gait recovery and disturbances using the CatWalk System, at baseline and monthly after DMM as previously published.

Assessment of joint pain via static weight bearing of hind paws by incapacitance testing 2X/wk starting 2 weeks before DMM using Incapacitance Meter, and data analyzed using

Rupatadine drug combinations

| | | | | | |
|---|---|---|---|---|---|
| Drug | Type | Target | Dosage and route | Bioavailability | Dev. Status |

Small molecule agonists of nuclear receptor proteins

| | | | | | |
|---|---|---|---|---|---|
| Fenofibrate | Fibrate | PPARα | 5 and 10 mg/kg oral | 60% | FDA- |
| | | | gavage qD | | approved |
| Gemfibrozil | Fibrate | PPARα | 20 mg/kg and 60 | 100% | FDA- |
| | | | mg/kg IP qD | | approved |
| Ciprofibrate | Fibrate | PPARα | 1 mg/kg and 2 mg/kg | | FDA- |
| | | | oral gavage qD | | approved |
| Bezafibrate | Fibrate | pan- | 5 or 10 mg/kg oral | 100% | FDA- |
| | | PPAR | gavage qD | | approved |
| Pioglitazone | Thiazolidinediones | PPARγ | 15 or 30 mg/kg orally | 83% | FDA- |
| | | | qD | | approved |

PAF inhibitors

| | | |
|---|---|---|
| Rupatadine | PAFR | |
| TSI-01 | LPCAT2 | investigational |
| WEB2086 | PAFR | |
| CV-3988 | PAFR | |

Lipid-lowering drugs

| | | | | | |
|---|---|---|---|---|---|
| Ezetimibe | Cholesterol | NPC1 | 50 mg/kg and 100 | variable | FDA- |
| | absorption | | mg/kg orally qD | | approved |
| | inhib. | | | | |
| Cholestyramine | Bile acid | | 50 mg/kg and 100 | | FDA- |
| | sequestrant | | mg/kg orally qD | | approved |

Small molecule antagonists of pathogenic receptors

| | | | | |
|---|---|---|---|---|
| CU-CPT22 | TLR2 | TLR2 | 5 mg/kg and 10 | Investigational |
| | antagonist | | mg/kg IP qD | |
| AP5055 | CD36 | CD36 | 5 mg/kg and 10 | Investigational |
| | antagonist | | mg/kg IP qD | |

Biguanide class of anti-diabetic agents

| | | | |
|---|---|---|---|
| Metformin | Biguanide | 1 mg/d | FDA- |
| | | | approved |
| Phenformin | Biguanide | | |

Small molecule bile acids

| | | |
|---|---|---|
| Ursodeoxycholic | Bile acid | FDA- |
| acid | | approved |
| Obeticholic acid | Bile acid | FDA- |
| | | approved |
| Deoxycholic acid | Bile acid | FDA- |
| | | approved |

### SEDACOM Software

X-ray & microCT. Performed at baseline & monthly using X-RAD 320 and vivaCT microCT scanners. A blinded scorer assesses degenerative changes, osteophyte formation, and subchondral sclerosis.

Histologic analysis. Histologic analysis of mouse stifle joints occurs 20-wks after surgery. Cartilage degeneration is assessed through scoring of Safranin-O stained sections and synovitis and osteophyte formation assessed through scoring of H&E stained sections. Significance was assessed by unpaired 2-tailed Mann-Whitney U test, with *P<0.05, **P<0.01, ***P<0.001.

Mechanistic studies: (i) Immunostaining, (ii) qPCR and/or bulk RNAseq transcriptional analysis, and (iii) biomarker analyses to characterize the mechanisms by which efficacious candidate therapeutics prevent OA. Upon identification of drugs that promote gait recovery and prevent OA, mechanistic studies will be performed. qPCR and/or bulk RNAseq will be performed on RNA isolated from synovial linings to analyze key transcriptional pathways to characterize how the effective drugs modulate lipid metabolism. ELISA will be performed on synovial fluids (as performed several of our prior publications) to analyze levels of LDLs & ox-LDLs. We will characterize the biomarkers in serum from mice that are protected vs. develop OA.

Power Calculations. Based on the aggregate variability of “OA Score” in DMM mice from 5 recent DMM experiments, the following parameters were input into the PS Power and Sample Size Calculator (W. D. Dupont and W. D. Plummer, Dept Biostatistics, Vanderbilt; Version 3.0.43): sample-size determination, unpaired: a=0.05 (Type I error), 8 25 (difference in mean OA Score between groups), o=15 (within group std deviation of the OA Score), m=1 (ratio of control to experimental mice), and power=0.8. The software calculated a minimum sample size of 7-8 mice per arm to detect a true difference of 25 in OA Score with a significance level of 0.05, and as a result we will use a group size of 10 mice per treatment subgroup.

Statistical analysis. We analyze (i) histological metrics (cartilage degeneration, proteoglycan loss, synovitis, osteophyte formation); (ii) radiographic metrics (X-ray, microCT); and (iii) mechanistic changes (qPCR, ELISA) in mice treated with lipid lowering drugs vs. vehicle using two-tailed unpaired t-tests. For comparisons involving >2 groups, we will use ANOVA followed by Dunnett's post-hoc test. Functional gait analysis: We analyze two-group stride pattern means by two-tailed unpaired Student's t-test, and multiple comparisons by one-way ANOVA followed by Dunnett's post-hoc test, as we previously did. Stride analysis, weight bearing, and additional “exploratory” analyses, are performed.

### Example 2

### Rupatadine Plus Celecoxib Small Molecule Combination Reduced Pain and Inflammatory Mediator Gene Expression in Human OA Synovial Tissue Organoid Stimulation Assays

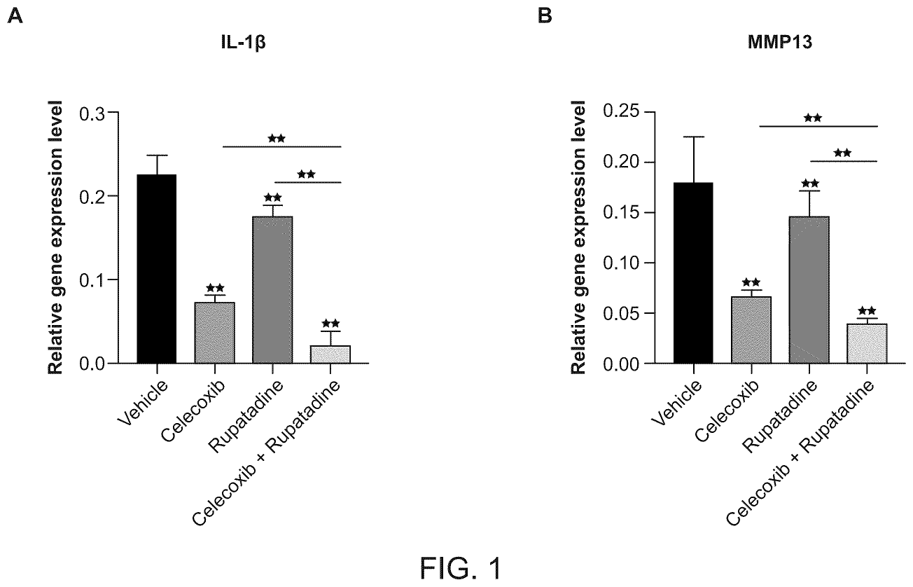

Human OA synovium was obtained at the time of arthroplasty and cultured overnight in tissue culture medium with human OA synovial fluid. The arthroplasty synovium was then washed with media, cut into approximately 2 mm pieces, and individual organoid pieces further stimulated for 24 hr with human OA synovial fluid in the presence of the test small molecule compounds and combinations including: vehicle control; celecoxib 1 μM; rupatadine 1 μM; or celecoxib 1 μM+rupatadine 1 μM. The organoids were then harvested, total RNA extracted, reverse transcription performed, followed by qPCR to quantitate levels of RNA encoding the pain/inflammatory mediator IL-1β and the degradative mediator MMP13, both of which were synergistically reduced by treatment with the combination of rupatadine+celecoxib (FIG. 1). Significance was assessed by t test, *P<0.05, **P<0.01.

### Example 3

### Rupatadine Plus Celecoxib Small Molecule Combination Synergistically Reduced Pain in the Mouse DMM Model for OA

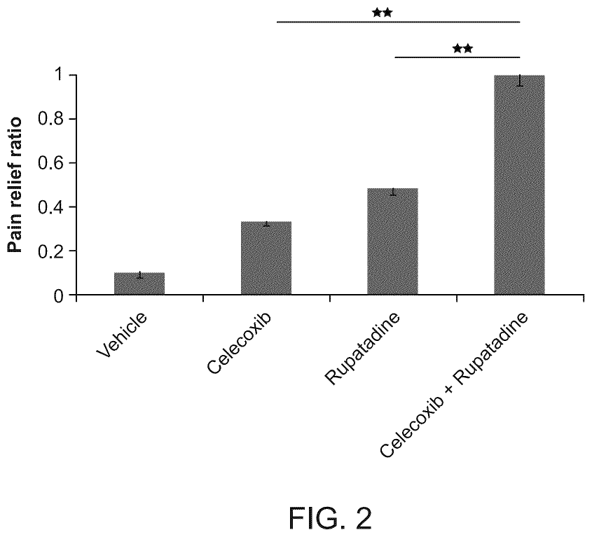

C57BI/6 mice at 5 months of age were subjected to surgical destabilization of the medial meniscus (DMM) or sham surgery on the contralateral joint, and starting 1 week following DMM surgery groups 10 mice were treated by daily oral gavage with vehicle control/day; or Rupatadine 20 μg/day; or Celecoxib 100 μg/day; or Rupatadine 20μg/day+Celecoxib 100 μg/day. After 11 weeks of treatment and while on ongoing treatment, mice were subjected to static weight bearing analysis of their hind paws which represents a functional pain outcome measurement. When mice experience joint pain, the affected limb and paw exhibit less weight bearing in this assay. For each mouse, 30 measurements for the simultaneous weight bearing of both hind paws, including both the surgical DMM limb paw and the sham-surgical comparator limb paw, were made. Mean weight bearing loads were calculated for each hind paw of each mouse, then the mean weight bearing loads were determined for each experimental group, and then based on these data the pain relief ratio (amount of increased load the surgical DMM limb paw was able to bear) was calculated. Treatment with the Rupatadine plus Celecoxib small molecule combination synergistically reduced pain in the mouse DMM model for OA, as compared to treatment with Rupatadine alone or Celecoxib alone (FIG. 2). Significance was assessed by t test, *P<0.05, **P<0.01. Synergy determined based on the following calculation: ([Celecoxib+Rupatadine]−[Vehicle])/(([Celecoxib]−[Vehicle])+([Rupatadine]−[Vehicle])))>1; with value>1 representing synergy.

### Example 4

Rupatadine Plus Celecoxib Small Molecule Combination Synergistically Reduced Cartilage Degeneration in the Mouse DMM Model for OA

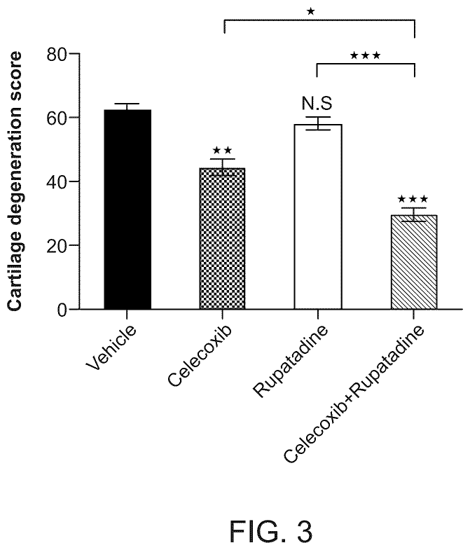

C57BI/6 mice at 5 months of age were subjected to surgical destabilization of the medial meniscus (DMM) or sham surgery on the contralateral joint, and starting 1 week following DMM surgery groups 10 mice were treated by daily oral gavage with vehicle control/day; or Rupatadine 20 μg/day; or Celecoxib 100 μg/day; or Rupatadine 20 μg/day+Celecoxib 100 μg/day. After 12 weeks of treatment, mice were sacrificed, and the stifle joints harvested for histologic analysis, and safranin-O stained joint tissue sections were scored by a blinded examiner for the degree of cartilage degeneration. Treatment with the small molecule combination of Rupatadine plus Celecoxib exhibited synergistic efficacy in treating mice to protect against cartilage degeneration, as compared to treatment with either rupatadine or celecoxib alone (FIG. 3). Significance was assessed by unpaired 2-tailed Mann-Whitney U test, with *P<0.05, **P<0.01, ***P<0.001, N.S. non-significant.

### Example 5

### Cetirizine Plus Celecoxib Small Molecule Combination Synergistically Reduces Cartilage Degeneration in the Mouse DMM Model for OA

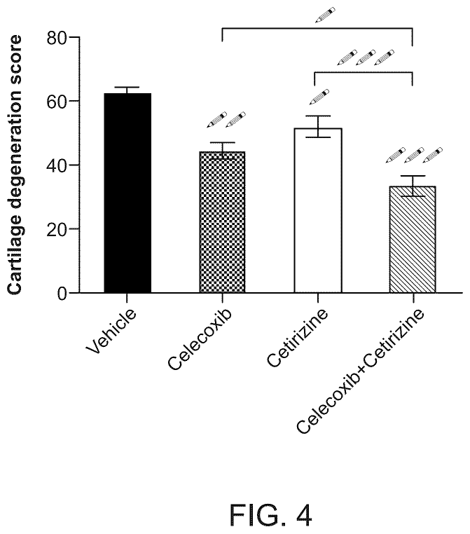

C57BI/6 mice at 5 months of age were subjected to surgical destabilization of the medial meniscus (DMM) or sham surgery on the contralateral joint, and starting 1 week following DMM surgery groups 10 mice were treated by daily oral gavage with vehicle control/day; or Cetirizine 20 μg/day; or Celecoxib 100 μg/day; or Cetirizine 20μg/day+Celecoxib 100 μg/day. After 12 weeks of treatment, mice were sacrificed, and the stifle joints harvested for histologic analysis, and safranin-O stained joint tissue sections were scored by a blinded examiner for the degree of cartilage degeneration. Treatment with the small molecule combination of Cetirizine plus Celecoxib exhibited synergistic efficacy in treating mice to protect against cartilage degeneration, as compared to treatment with either Cetirizine or Celecoxib alone (FIG. 4). Significance was assessed by unpaired 2-tailed Mann-WhitneyU test, with *P<0.05, **P<0.01, ***P<0.001.

### Example 6

### Rupatadine Plus Celecoxib Small Molecule Combination Synergistically Reduced Pain, Inflammatory and Degradative Mediator Gene Expression in Human OA Synoviocytes In Vitro

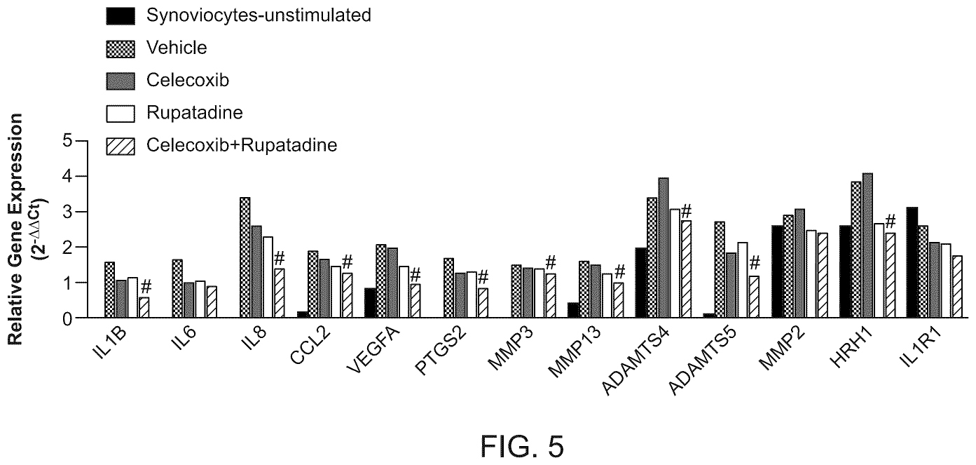

Human synoviocytes were isolated from remnant knee tissue obtained at the time of total knee replacement (arthroplasty). Remnant knee synovial lining was digested with collagenase IV to isolate synoviocytes, and synoviocytes cultured in vitro in 24 well plates at 2×10⁵/well. The synoviocytes were treated with PBS vehicle; or 50 nM Celecoxib; or 50 nM Rupatadine; or 50 nM Celecoxib plus 50 nM Rupatadine; and 30 minutes later the synoviocytes were stimulated with human chondrocyte debris. After 24 hours of stimulation, the synoviocytes were collected and total RNA isolated. Quantitative polymerase chain reaction (qPCR) was performed on the isolated RNA to measure levels of RNA encoding the indicated genes. Treatment of synoviocytes with the Rupatadine plus Celecoxib combination synergistically reduced gene expression of multiple pain, inflammatory and degradative mediators including IL1β, IL8, VEGFα, MMP3, MMP13, ADAMTS4, ADAMTS5 and HRH1 (FIG. 5). Synergy determined based on the following calculation: (([Vehicle]−[Celecoxib+Rupatadine combination])/([Vehicle−Celecoxib alone]+[Vehicle−Rupatadine alone]))>1; and synergistic inhibition is indicated by #s.

### Example 7

### Cetirizine Plus Celecoxib Small Molecule Combination Synergistically Reduced Pain, Inflammatory and Degradative Mediator Gene Expression in Human OA Synoviocytes In Vitro

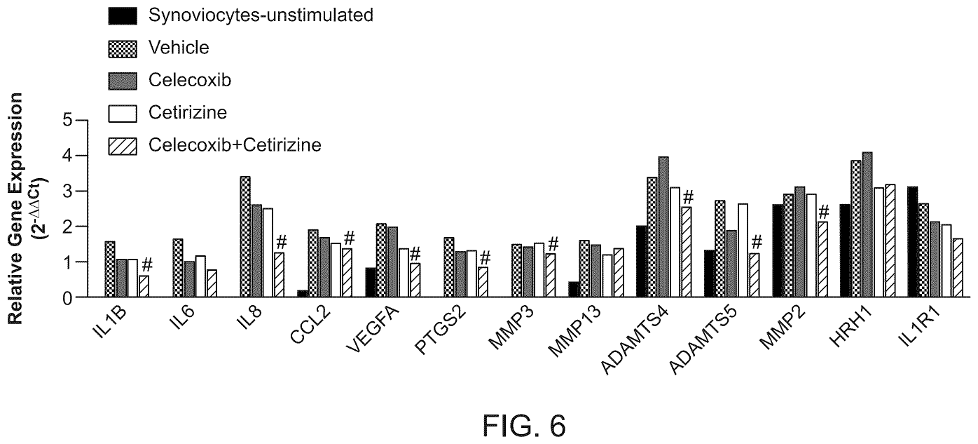

Human synoviocytes were isolated from remnant knee tissue obtained at the time of total knee replacement (arthroplasty). Remnant knee synovial lining was digested with collagenase IV to isolate synoviocytes, and synoviocytes cultured in vitro in 24 well plates at 2×10⁵/well. The synoviocytes were treated with PBS vehicle; or 50 nM Celecoxib; or 50 nM Cetirizine; or 50 nM Celecoxib plus 50 nM Cetirizine; and 30 minutes later the synoviocytes were stimulated with human chondrocyte debris. After 24 hours of stimulation, the synoviocytes were collected and total RNA isolated. Quantitative polymerase chain reaction (qPCR) was performed on the isolated RNA to measure levels of RNA encoding the indicated genes. Treatment of synoviocytes with the Cetirizine plus Celecoxib combination synergistically reduced gene expression of multiple pain, inflammatory and degradative mediators including IL1β, IL8, CCL2, VEGFα, PTGS2, MMP3, ADAMTS4, ADAMTS5, and MMP2 (FIG. 6). Synergy determined based on the following calculation: (([Vehicle]−[Celecoxib+Cetirizine combination])/([Vehicle−Celecoxib alone]+[Vehicle−Cetirizine alone]))>1; and synergistic inhibition is indicated by #s.

### Example 8

### Rupatadine Plus Celecoxib Small Molecule Combination Synergistically Reduced Pain, Inflammatory and Degradative Mediator Gene Expression in Human OA Chondrocytes In Vitro

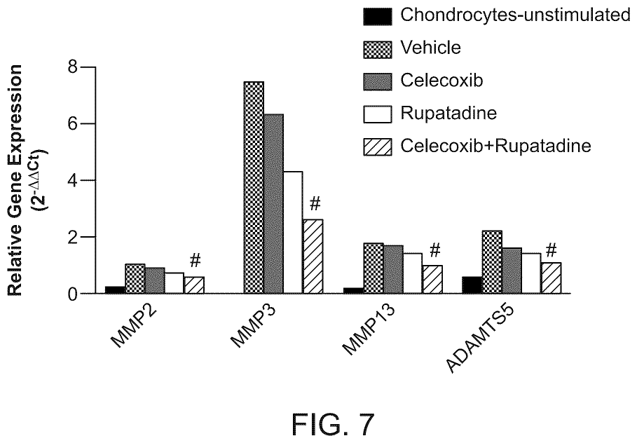

Human chondrocytes were isolated remnant knee tissue obtained at the time of total knee replacement (arthroplasty). Remnant knee cartilage was digested with collagenase IV to isolate chondrocytes, and chondrocytes cultured in vitro in 24 well plates at 2×10⁵/well. The chondrocytes were treated with PBS vehicle; or 50 nM Celecoxib; or 50 nM Rupatadine; or 50 nM Celecoxib plus 50 nM Rupatadine; and 30 minutes later the chondrocytes were stimulated with human chondrocyte debris. After 24 hours of stimulation, the chondrocytes were collected and total RNA isolated. Quantitative polymerase chain reaction (qPCR) was performed on the isolated RNA to measure levels of RNA encoding the indicated genes. Treatment of chondrocytes with the Rupatadine plus Celecoxib combination synergistically reduced gene expression of OA degradative mediators including MMP2, MMP3, MMP13, and ADAMTS5 (FIG. 7). Synergy determined based on the following calculation: (([Vehicle]−[Celecoxib+Rupatadine combination])/([Vehicle−Celecoxib alone]+[Vehicle-Rupatadine alone]))>1; and synergistic inhibition is indicated by #s.

### Example 9

### Cetirizine Plus Celecoxib Small Molecule Combination Synergistically Reduced Pain, Inflammatory and Degradative Mediator Expression in Human OA Chondrocytes In Vitro

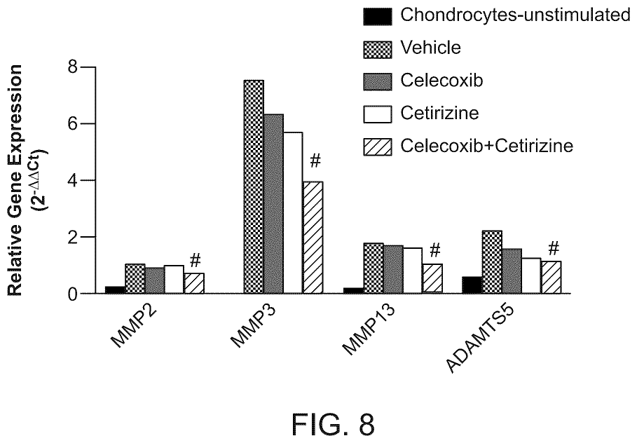

Human chondrocytes were isolated remnant knee tissue obtained at the time of total knee replacement (arthroplasty). Remnant knee cartilage was digested with collagenase IV to isolate chondrocytes, and chondrocytes cultured in vitro in 24 well plates at 2×10⁵/well. The chondrocytes were treated with PBS vehicle; or 50 nM Celecoxib; or 50 nM Cetirizine; or 50 nM Celecoxib plus 50 nM Cetirizine; and 30 minutes later the chondrocytes were stimulated with human chondrocyte debris. After 24 hours of stimulation, the chondrocytes were collected and total RNA isolated. Quantitative polymerase chain reaction (qPCR) was performed on the isolated RNA to measure levels of RNA encoding the indicated genes. Treatment of chondrocytes with the Rupatadine plus Celecoxib combination synergistically reduced gene expression of OA degradative mediators including MMP2, MMP3, MMP13, and ADAMTS5 (FIG. 8). Synergy determined based on the following calculation: (([Vehicle]−[Celecoxib+Cetirizine combination])/([Vehicle-Celecoxib alone]+[Vehicle−Cetirizine alone]))>1; and synergistic inhibition is indicated by #s.

### Example 10

Rupatadine Plus Fenofibrate Small Molecule Combination Reduced Inflammatory Lipid PAF Biosynthesis Enzyme (Ipcat2) Gene Expression as Well as PAF Receptor (ptafr) Gene Expression in Human OA Synovial Tissue Organoid Stimulation Assays

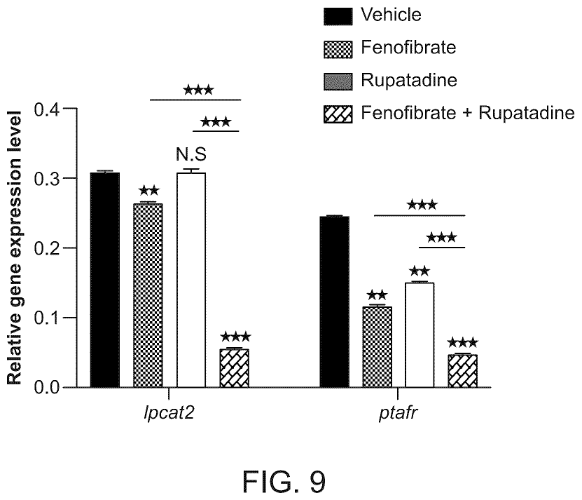

Human OA synovium was obtained at the time of arthroplasty and cultured overnight in tissue culture medium with human OA synovial fluid. The arthroplasty synovium was then washed with media, cut into approximately 2 mm pieces, and individual organoid pieces further stimulated for 24 hr with human OA synovial fluid in the presence of the test small molecule compounds and combinations including: vehicle control; rupatadine 1 μM; fenofibrate 1 μM; or rupatadine 1 μM+fenofibrate 1 μM. The organoids were then harvested, total RNA extracted, reverse transcription performed, followed by qPCR to quantitate levels of RNA encoding the inflammatory lipid PAF biosynthesis enzyme (Ipcat2) gene expression as well as PAF receptor (ptafr) gene expression. Both the PPARα agonist fenofibrate and the H1 receptor inhibitor and PAF antagonist rupatadine decreased Ipcat2 gene expression as well as ptafr gene expression, and the combination of fenofibrate plus rupatadine synergistically decreased expression of Ipcat2 (FIG. 9). Significance was assessed by t test, *P<0.05, **P<0.01, ***P<0.001. Synergy determined based on the following calculation: ([Vehicle]−[Fenofibrate+Rupatadine])/(([Vehicle]−[Fenofibrate])+([Vehicle]−[Rupatadine])))>1; with value>1 representing synergy.

### Example 11

### Rupatadine Plus Fenofibrate Small Molecule Combination Reduced Pain and Inflammatory Mediator Expression in Human OA Synovial Tissue Organoid Stimulation Assays

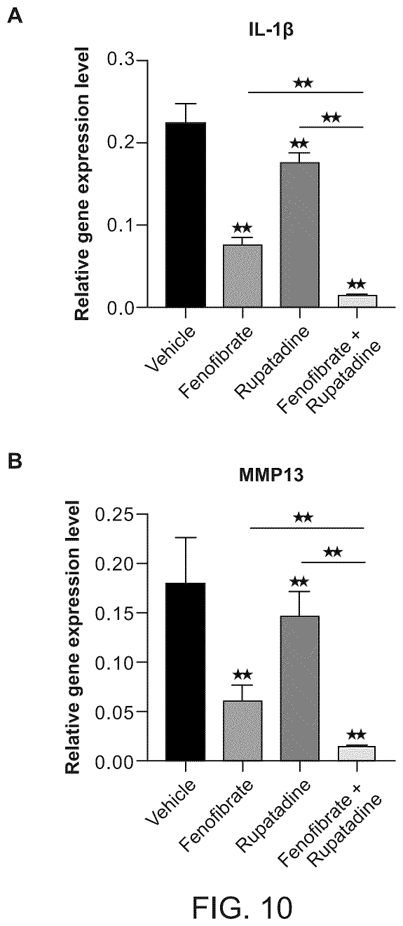

Human OA synovium was obtained at the time of arthroplasty and cultured overnight in tissue culture medium with human OA synovial fluid. The arthroplasty synovium was then washed with media, cut into approximately 2 mm pieces, and individual organoid pieces further stimulated for 24 hr with human OA synovial fluid in the presence of the test small molecule compounds and combinations including: vehicle control; rupatadine 1 μM; fenofibrate 1 μM; or rupatadine 1 μM+fenofibrate 1 μM. The organoids were then harvested, total RNA extracted, reverse transcription performed, followed by qPCR to quantitate levels of RNA encoding the pain/inflammatory mediator IL-1b and the degradative mediator MMP13 (FIG. 10). Significance was assessed by ttest, *P<0.05, **P<0.01.

### Example 12

### Rupatadine Plus Fenofibrate Drug Combination Prevented Development Of OA In The DMM Mouse Model

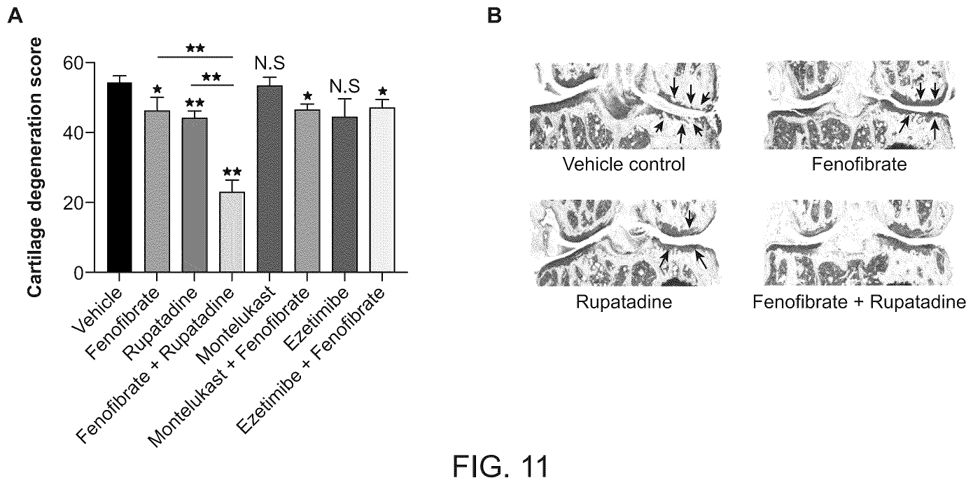

At 5 months of age, C57BI/6 mice underwent surgical destabilization of the medial meniscus (DMM) or sham surgery on the contralateral joint, and starting 1 week following DMM mice were treated by oral gavage with vehicle control; rupatadine 20 μg; fenofibrate 40 μg; or rupatadine 20 μg+fenofibrate 40 μg; montelukast 25 μg; montelukast 25 μg+fenofibrate 40 μg; ezetimibe 25 μg; ezetimibe 25 μg+fenofibrate 40 μg; for 3 months. Mice were then sacrificed, and the stifle joints harvested for histologic analysis. (A) Safranin-O stained joint tissue sections were scored by a blinded examiner for the degree of cartilage degeneration. (B) Representative safranin-O stained sections from each group are presented. Both the PPARα agonist fenofibrate and the H1 receptor inhibitor and PAF antagonist rupatadine decreased the severity of OA following DMM in mice, and the combination of fenofibrate plus rupatadine synergistically prevented development of OA (FIG. 11). Significance was assessed by unpaired 2-tailed Mann-Whitney U test, with *P<0.05, **P<0.01, ***P<0.001. Synergy determined based on the following calculation: ([Vehicle]−[Fenofibrate+Rupatadine])/(([Vehicle]−[Fenofibrate])+([Vehicle]−[Rupatadine])))>1; with value>1 representing synergy.

### Example 13

### The Combination of Rupatadine+Celecoxib Reduces Pain in OA

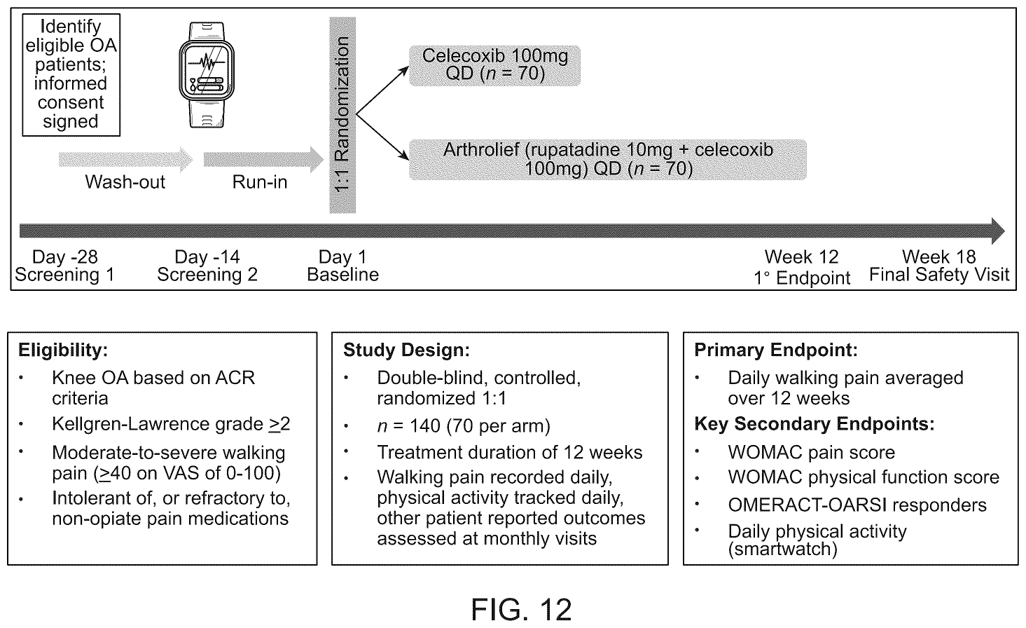

A clinical trial is run (FIG. 12) in which human OA patients are enrolled based on having (i) knee OA based on American College of Rheumatology (ACR) criteria, (ii) Kellgren-Lawrence grade ≥2 on X-ray of the knee, (iii) moderate-to-severe walking pain (≥40 on the visual analog scale [VAS] of 0-100), and (iv) intolerant of, or refractory to, non-opiate pain medications. After a 2-week run into assess baseline pain, patients are randomized 1:1 to treatment with celecoxib 100 mg/day vs. celecoxib 100 mg/day+rupatadine 10 mg/day. Walking pain is recorded daily, along with tracking of physical activity daily. Other patient reported outcomes are assessed at monthly evaluations. The Primary Endpoint is daily walking pain averaged over 12 weeks. Secondary endpoints include (i) Western Ontario and McMaster Universities Arthritis Index (WOMAC) pain scores, (ii) WOMAC physical function score, (iii) OMERACT-OARSI responder score, and (iv) daily physical activity. Over the 12-week in-life dosing period knee OA patients randomized to celecoxib 100 mg/day+rupatadine 10 mg/day report statistically less daily walking pain, statistically lower WOMAC pain scores, statistically increased WOMAC physical function scores, and/or statistically increased daily activity as compared to the patients randomized to celecoxib 100 mg/day.

### Example 14

The combination of rupatadine+fenofibrate reduces OA disease progression.

A clinical trial is run in which human OA patients are enrolled based on having (i) knee OA based on American College of Rheumatology (ACR) criteria, (ii) Kellgren-Lawrence grade ≥2 on X-ray of the knee, (iii) moderate-to-severe walking pain (>40 on the visual analog scale [VAS] of 0-100), and (iv) synovitis on gadolinium-enhanced MRI (Gd-MRI) of the knee. Baseline MRI with gadolinium contrast enhancement of the affected OA knee is obtained. After a 2-week run into assess baseline pain, patients are randomized 1:1 to treatment with fenofibrate 150 mg/day vs. fenofibrate 150 mg/day+rupatadine 10 mg/day vs. rupatadine 10 mg/day for 1 year. Walking pain is recorded daily, along with tracking of physical activity daily. Other patient reported outcomes are assessed at monthly evaluations. At 1 year a MRI with gadolinium contrast enhancement of the affected OA knee is obtained. The Primary Endpoint is change in knee cartilage volume by MRI at 1 year. Secondary endpoints include (i) proportion of subjects treated with fenofibrate 150 mg/day+rupatadine 10 mg/day achieving meaningful improvement in synovitis based on a reduction in the synovitis score (Guermazi et al., Ann Rheum Dis. 2011 70(5): 805-11. PMID: 21187293) as measured by Gd-MRI by greater than 4 points; (ii) daily walking pain averaged over 52 weeks; (iii) change from baseline to months 1, 3, 6, 9 and 12 in the WOMAC pain subscale, (iv) change from baseline to months 1, 3, 6, 9 and 12 in the WOMAC function subscale; change from baseline to months 1, 3, 6, 9 and 12 in the Patient's Global VAS; (v) analysis of efficacy data using the OMERACT-OARSI Responder Index (Onel et al, Clin Drug Investig. 2008; 28(1): 37-45. PMID: 18081359). Over the 12-month in-life dosing period knee OA patients randomized to fenofibrate 150 mg/day+rupatadine 10 mg/day are found to have statistically less cartilage volume loss on knee MRI, statistically reduced synovitis on knee!!0bMRI, statistically lower daily walking pain, statistically lower WOMAC pain scores, statistically increased WOMAC physical function scores, and/or statistically increased daily activity as compared to the patients randomized to fenofibrate 150 mg/day or rupatadine 10 mg/day.

### Example 15

### Inflammatory Lipids in Osteoarthritis

The role of inflammatory lipids in the development of osteoarthritis (OA) was investigated. Many humans develop OA and/or sustain joint injuries that lead to joint dysfunction and post-traumatic OA (PTOA). Existing treatments are ineffective in preventing or treating OA. Increasing evidence implicates a key role for innate immune inflammation in the pathogenesis of OA. Understanding the underlying inflammatory mechanisms provides biomarkers and therapeutics that can transform care for military personnel, Veterans, and civilians with OA and/or joint injuries.

OA is associated with diseases in which serum levels of LDLs and triglycerides are elevated. Further, serum levels of modified LDLs (e.g. oxLDL and acLDL) positively correlate with OA severity. Modified LDLs promote inflammatory responses and production of matrix metalloproteinases which contribute to cartilage destruction in OA and RA. Mice deficient for genes involved in uptake and transport of LDLs, including APOE, LDLR, and CAV1 develop more severe DMM-induced OA. Mice genetically deficient for the lipid metabolism activator PPARα developed severe OA, while treatment with the PPARα agonist fenofibrate reduced the severity of OA. Mice deficient for the oxLDL-binding scavenger receptor CD36 were protected against OA. Mice deficient for the oxLDL-binding immune activating receptor TLR2 were also protected against OA. Together, our findings suggest critical roles for PPARα-dysregulated lipid metabolism and inflammatory lipids in OA pathogenesis.

Altered lipid metabolism and increased levels of inflammatory lipids, including oxLDLs, acLDLs and plasmalogens, may promote the development of OA. Specifically, reduced PPARα activity in synovial macrophages, fibroblasts and chondrocytes may result in the accumulation of extracellular inflammatory lipid precursors; due to reduced cellular uptake of inflammatory lipid precursors, these precursors become modified into pro-inflammatory forms including oxLDL and acLDL that bind CD36, TLR2, and/or TLR4 to activate synovial macrophages, fibroblasts and chondrocytes to produce pro-inflammatory mediators; and inflammatory phospholipids such as plasmalogen activate synovial macrophages, fibroblasts and chondrocytes to produce pro-inflammatory and degradative mediators. Treatment with PPARα agonists may prevent and treat OA through reducing inflammatory lipid production and LDL modification.

i) Lipoproteins and modified LDLs are complex particles comprised of a core of hydrophobic molecules including cholesterol esters and triglycerides, surrounded by apolipoproteins (e.g. ApoAI, ApoE) and phospholipids which facilitate transport of the insoluble components. Modifications of lipoproteins are associated with multiple diseases and can alter the structure and function of the lipoproteins. Oxidized LDL (oxLDL) arises as a result of oxidation of either amino acids of the apolipoproteins, or of the attached lipids by ROS. Serum levels of oxLDL are positively correlated with OA severity, and oxLDL can stimulate MMP production which can contribute to cartilage destruction. LDLs can also be acetylated (acLDL) and similarly can be taken up by RA and OA synoviocytes.

ii) Inflammatory Phospholipids. Phospholipids are the major components of cell membranes and contribute to the lubricating properties of synovial fluids. Platelet-activating factor (PAF) PAF is a lipid mediator that is well-known for its ability to cause platelet aggregation, inflammation, and allergic response at very low concentrations. PAF is synthesized primarily through lipid remodeling. A phospholipid (often phosphatidyl choline) is converted to a lyso-PC (LPC) intermediate, which is then converted to PAF by LPC acetyltransferase (LPCAT). PAF signals through the Platelet-activating factor receptor (PAFR), which is expressed on the surface of many cell types. PAFR is a member of the G protein-coupled receptor superfamily. PAF is produced by multiple cell types including mast cells, basophils, neutrophils, eosinophils, fibroblasts, platelets, endothelial cells, and cardiac muscle cells and is known to play an important role in inflammatory, thrombotic and allergic conditions. Plasmalogens, a group of phospholipids with vinyl-ether bonds, include phosphatidylcholine (PC P) and phosphatidylethanolamine (PE P) and have multiple molecular roles in modulating membrane fluidity as well as antioxidant function protecting against ROS. Studies demonstrated that alterations of synovial fluid phospholipid composition are associated with OA and that PE P plasmalogens are increased in OA synovial fluids. These studies suggest that changes in phospholipid and plasmalogen composition might contribute to the pathogenesis of OA.

PPARα. PPARα is a ligand-activated transcription factor belonging to the class I nuclear receptor family. Natural ligands of PPARα include polyunsaturated fatty acids, prostaglandins and leukotrienes. Activation of PPARα promotes anti-inflammatory responses including inhibition of metalloproteinases, nuclear translocation of NF-κB, and upregulates IL-1 receptor antagonist which counteracts IL-1β function. Therapeutic activation of PPARα function, through agonists such as fibrates are effective in treating hypercholesterolemia and promote the degradation of fatty acids. Treatment of OA cartilage with a PPARα agonist resulted in lower levels of known OA inflammatory and degradative mediators. oxLDL stimulation of innate inflammation. There is strong evidence for a connection between LDL accumulation and activation of the innate immune system. Macrophages detect oxLDLs through scavenger receptors expressed, such as CD36, on the cell surface and take up oxLDLs through phagocytosis, which promotes their differentiation into foam cells and activation of pro-inflammatory responses. Modified LDLs also act as ligands for TLRs, which are expressed on the surface and in the lysosomes of macrophages, with the later activated following macrophage phagocytosis of oxLDL. Evidence suggests that signaling through CD36 and oxLDL uptake may be enhanced by activation of PAFR or TLR2. In atherosclerosis, accumulation of oxLDLs in arterial walls leads to activation of

Macrophages in OA. Macrophage infiltrates are present in the synovial tissues of individuals with OA, and have been shown to contribute to OA pathogenesis. Uptake of modified LDLs by macrophages results in the triggering of pro-inflammatory responses, transformation into foam cells and can lead to changes in plasmalogen levels and structure. Synovial macrophages may respond to the presence of oxLDL and acLDL and promote expression of inflammatory mediators in OA.

Mast cells in OA. Mast cells and several mast cell mediators are present in the synovium from individuals with OA, and also contribute to chronic inflammation in allergic disease. We recently demonstrated that IgE-mediated degranulation of mast cells, and the mast cell-specific product, tryptase promotes inflammation and joint destruction in OA. In allergic disease, where mast cells have been most extensively studied, these mediators promote chronic allergic inflammation which, if sustained, results in long-term tissue damage and remodeling, which is similarly seen in OA. Mast cells are known to regulate lipid metabolism and vascular events during atherosclerosis. Mast cells can induce degranulation in the presence of ox-LDL and PAF, and can promote uptake of ox-LDL by macrophages. Given the presence of mast cells in the synovium of osteoarthritic joints and their role in promoting OA, we hypothesize that increased levels inflammatory lipids including PAF and ox-LDLs promote mast cell activation and the release of mediators that contribute to joint destruction in OA.

Destabilization of the medial meniscus (DMM) mouse models of post-traumatic OA. In humans, loss of meniscal integrity diminishes the ability of the joint to disperse load and absorb shock, and meniscal degeneration is associated with the development of OA. Surgical ligation of the ligament stabilizing the medial meniscus in the stifle (knee) joint in mice leads to OA-like pathology. The DMM model reflects mild-to-moderate OA, and is appropriate for the study of OA resulting from meniscal tearing. The DMM model shares multiple histological features with human OA, including proteoglycan loss, cartilage loss, & osteophyte formation.

PPARα function prevents development of OA. Activation of PPARα, a nuclear receptor involved in the metabolism of fatty acids, prevents inflammatory responses in chondrocytes in OA. We investigated the role of PPARα in the pathogenesis of OA by analyzing OA severity in mice deficient for PPARα (ppara^{-/-}) following DMM. ppara^{-/-} mice demonstrated significantly more severe cartilage degradation following DMM as compared to WT mice. We further tested the ability of fenofibrate, a synthetic PPARα agonist, to prevent OA by treating WT mice with either 10 mg/kg fenofibrate or vehicle for 12 wks following DMM. Fenofibrate-treated mice exhibited significantly decreased cartilage degradation as compared to vehicle-treated mice. These data indicate that activation of PPARα ameliorates OA following joint injury. Consistent with these findings, we demonstrated that PPARA gene expression is decreased in the synovial tissues of individuals with OA, as compared to healthy and RA, while a previous study demonstrated that PPARA gene expression is decreased in OA chondrocytes.

Apolipoproteins play a key roles in development of PTOA. Apolipoproteins are detected in human OA synovial fluids. First-generation mass spectrometry analyses of OA synovial fluids identified multiple lipid transport proteins including ApoA-I, ApoA-II, ApoA-IV, ApoE, and LRP1.

APOE gene expression is decreased in aged mice and following joint injury. Activation of PPARα results in lowering of serum triglyceride levels and promotes metabolism of LDLs. As ApoE is a lipid carrier protein that comprises certain types of LDLs, we analyzed gene expression in synovial tissues of young (2 wk) wild-type (WT) mice; old (12 mo) WT mice; and WT mice with DMM-induced OA (old WT DMM). This analysis revealed that APOE expression is decreased in older mice and following joint injury. These results suggest ApoE expression is dysregulated in OA.

Defects in LDL uptake promote OA development. We further analyzed the role of ApoE in OA by surgically inducing DMM in WT and ApoE-deficient (apoe^{-/-}) mice. ApoE-deficient mice developed significantly more severe cartilage degradation, suggesting a protective role for ApoE in OA. Low-density lipoprotein receptor (LDLR) is a cell surface receptor that mediates endocytosis of LDLs, thereby maintaining plasma levels of LDL. To evaluate the role of LDLR in OA, we induced DMM in WT and LDLR-deficient (ldlr^{-/-}) mice. LDLR-deficient mice developed more severe cartilage degradation compared to WT, suggesting that LDLR is protective against OA. Caveolin 1 is a membrane protein important in LDL homeostasis that was previously implicated in OA pathogenesis. To analyze the role of caveolin1 in OA, we induced DMM in CAV1-deficient (cav1^{-/-}) mice. cav1^{-/-} mice had increased cartilage degradation following DMM as compared to wild-type (WT) mice. Chondrocytes derived from DMM joints of cav1^{-/-} mice expressed higher levels of inflammatory and degradative mediators as compared to those from WT.

Modified LDL uptake promotes OA. CD36 deficiency reduces severity of OA following DMM. CD36 is a scavenger receptor expressed on the surface of macrophages, platelets, and other cells that that triggers pro-inflammatory responses upon binding ox-LDL. To investigate the role of CD36 in OA, we induced DMM in CD36-deficient (cd36^{-/-}) mice. 20 wks after DMM, cd36^{-/-} mice had significantly reduced cartilage degeneration as compared to WT, indicating that CD36 contributes to the pathogenesis of OA.

TLR2 deficiency protects against OA following DMM. oxLDL triggers inflammatory responses in a TLR2-CD36, dependent manner. To test the role of TLR2 in OA, we induced DMM in tlr2^{-/-} mice. 20 wks after DMM, tlr2^{-/-} mice exhibited reduced cartilage degradation, suggesting that TLR2 promotes OA pathogenesis.

Acetylated LDL promotes pathogenic responses in FLS. Modified LDLs promote inflammation and are associated with OA severity. We tested whether acetylated LDL (acLDL) can promote pathogenic responses by stimulating FLS derived from the joints of wild-type mice with acLDL. qPCR quantification of mRNA of genes encoding known OA pathogenic mediators revealed that acLDL promotes the expression of multiple inflammatory and degradative mediators

Modified LDLs downregulate PPARA expression. We tested whether oxLDL or acLDL regulate PPARA expression. Stimulation of human OA synoviocyte and chondrocytes with either oxLDL or acLDL resulted in significantly decreased expression of PPARA.

Platelet-activating factor promotes OA pathogenesis. PAF present in OA and joint injury synovial fluids. To analyze a role for PAF in joint injury and OA, we quantified levels of PAF in synovial fluids of individuals with OA, ACL tear or DMT via ELISA.

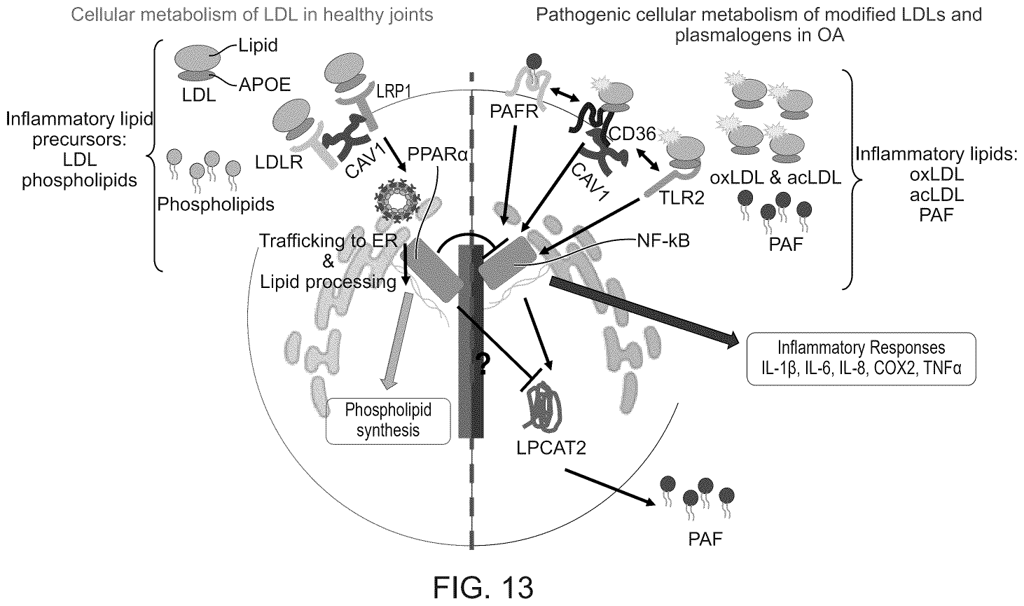

FIG. 13. presents a summary of these mechanistic findings, including the roles of PPARa and the production of PAF in response to inflammatory lipid stimulation.

All publications and patent documents cited herein are incorporated by reference in their entirety. To the extent the material incorporated by reference contradicts or is inconsistent with the present specification, the present specification will supersede any such material.

1. A method of reducing progression of an inflammatory disease or reducing pain associated with an inflammatory disease, the method comprising:
administering to an individual in need thereof, an effective dose of a combination of a selective histamine H1 receptor antagonist in combination with a second agent that provides for reduction of pain, inflammation, or inflammatory lipids.

2. The method of claim 1, wherein the inflammatory disease is selected from osteoarthritis, rheumatoid arthritis, juvenile rheumatoid arthritis, juvenile idiopathic arthritis, psoriatic arthritis, ankylosing spondylitis, arthritis, joint injury, primary dysmenorrhea, or another condition associated with pain.

3. (canceled)

4. The method of claim 1, wherein the selective histamine H1 receptor antagonist also blocks receptors of platelet-activating factor (PAF).

5. The method of claim 1, wherein the selective histamine HH1 1 receptor antagonist is rupatadine.

6. The method of claim 1, wherein the selective histamine H1 receptor antagonist is fexofenadine, cetirizine, loratadine, astemizole, ketotifen, mizolastine acrivastine, ebastine, bilastine, bepotastine, terfenadine, or quifenadine.

7. The method of claim 1, wherein the combination provides for one or both of a synergistic benefit in reducing pain and a synergistic benefit in reducing disease progression.

8. (canceled)

9. The method of claim 1, wherein the individual in need thereof is at a pre-clinical time point for the inflammatory disease.

10. The method of claim 1, wherein the individual in need thereof has an established inflammatory disease.

11. The method of claim 1, wherein the combination of agents is administered separately or is co-formulated.

12. (canceled)

13. The method of claim 1, wherein the administration of one or both agents is oral administration.

14. The method of claim 1, wherein the second agent is a COX-2 inhibitor, optionally wherein the COX-2 inhibitor is celecoxib.

15. (canceled)

16. The method of claim 14, wherein the co-formulated combination comprises rupadatine at a unit dose of 10 mg and celecoxib at a unit dose of 100 mg.

17. The method of claim 14, wherein the co-formulated combination comprises rupadatine at a unit dose of 5 mg and celecoxib at a unit dose of 100 mg.

18. The method of claim 1, wherein the second agent is a fibrate, optionally wherein the fibrate is fenofibrate.

19. (canceled)

20. The method of claim 1, wherein the second agent is a PPARD antagonist optionally wherein the PPARD antagonist is fenofibrate or TPST-1120.

21. (canceled)

22. The method of claim 1, wherein the second agent is a bile acid, optionally wherein the bile acid is ursodeoxycholic acid, obetadeoxycholic acid, obeticholic acid, or deoxycholic acid.

23. (canceled)

24. The method of claim 1, wherein the second agent is sertraline.

25. The method of claim 1, wherein the second agent is metformin.

26. The method of claim 1, wherein rupatadine is co-formulated with fexofenadine, cetirizine, loratadine, astemizole, ketotifen, mizolastine acrivastine, ebastine, bilastine, bepotastine, terfenadine, or quifenadine.

27. A pharmaceutical formulation for use in the method of claim 1.
